(19) United States
(12) Patent Application Publication
(10) Pub. No.: US 2024/0197382 A1
(43) Pub. Date: Jun. 20, 2024

(54) TREATMENT SYSTEMS AND METHODS FOR TREATING CELLULITE AND FOR PROVIDING OTHER TREATMENTS

(71) Applicant: Zeltiq Aesthetics, Inc, Pleasanton, CA (US)

(72) Inventors: Leonard DeBenedictis, Dubloin, CA (US); George Frangineas, JR., Fremont, CA (US); Kristine Tatsutani, Redwood City, CA (US); Linda Pham, Kensington, CA (US)

(21) Appl. No.: 18/469,396

(22) Filed: Sep. 18, 2023

Related U.S. Application Data
(63) Continuation of application No. 17/719,661, filed on Apr. 13, 2022, now abandoned.
Application No. 17/719,661 is a continuation of application No. 16/227,376, filed on Dec. 20, 2018, now abandoned.
Application No. 16/227,376 is a continuation of application No. 14/611,052, filed on Jan. 30, 2015, now abandoned.
(60) Provisional application No. 61/943,257, filed on Feb. 21, 2014.
(60) Provisional application No. 61/943,250, filed on Feb. 21, 2014.
(60) Provisional application No. 61/934,549, filed on Jan. 31, 2014.

(51) Int. Cl. A61B 18/02; A61B 18/00; A61B 90/00; A61F 7/00; A61H 1/00; A61K 31/045; A61K 31/047; A61N 7/00

(52) U.S. Cl. CPC A61B 18/02; A61B 18/0206; A61B 90/04; A61F 7/00; A61F 7/007; A61H 1/006; A61H 1/008; A61K 31/045; A61K 31/047; A61N 7/00; A61B 2018/00291; A61B 2018/00464; A61B 2018/00714; A61B 2018/00791; A61B 2018/00875; A61B 2018/00994; A61B 2018/0237; A61B 2018/0262; A61B 2090/0463; A61B 2090/065; A61F 2007/0003; A61F 2007/0004; A61F 2007/0019; A61F 2007/0036; A61F 2007/0045; A61F 2007/0047; A61F 2007/0052; A61F 2007/0056; A61F 2007/0075; A61F 2007/0087; A61F 2007/0093; A61F 2007/0096

(57) ABSTRACT

Treatment systems, methods, and apparatuses for improving the appearance of skin or other target regions are described. Aspects of the technology are directed to improving the appearance of skin by tightening the skin, improving skin tone or texture, eliminating or reducing wrinkles, increasing skin smoothness, or improving the appearance sites with cellulite. Treatments can include cooling a surface of a patient's skin and detecting freezing in the cooled skin. The tissue can be cooled after the freeze event is detected so to maintain the frozen state of the tissue to improve the appearance of the treatment site.

### CROSS-REFERENCE TO RELATED APPLICATIONS

The present application is a continuation of U.S. patent application Ser. No. 17/719,661, filed on Apr. 13, 2022, which is a continuation of U.S. patent application Ser. No. 16/227,376, filed on Dec. 20, 2018, which is a continuation U.S. patent application Ser. No. 14/611,052, filed Jan. 30, 2015, which claims priority to U.S. Provisional Application Ser. No. 61/943,257, filed Feb. 21, 2014, U.S. Provisional Application Ser. No. 61/943,250, filed Feb. 21, 2014, and U.S. Provisional Application Ser. No. 61/934,549, filed Jan. 31, 2014, the disclosures of which are incorporated herein by reference in their entireties.

### INCORPORATION BY REFERENCE OF COMMONLY-OWNED APPLICATIONS AND PATENTS

The following commonly assigned U.S. Patent Applications and U.S. Patents are incorporated herein by reference in their entirety:

U.S. Patent Publication No. 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;

U.S. Pat. No. 6,032,675 entitled “FREEZING METHOD FOR CONTROLLED REMOVAL OF FATTY TISSUE BY LIPOSUCTION”;

U.S. Patent Publication No. 2007/0255362 entitled “CRYOPROTECTANT FOR USE WITH A TREATMENT DEVICE FOR IMPROVED COOLING OF SUBCUTANEOUS LIPID-RICH CELLS”;

U.S. Pat. No. 7,854,754 entitled “COOLING DEVICE FOR REMOVING HEAT FROM SUBCUTANEOUS LIPID-RICH CELLS”;

U.S. Pat. No. 8,337,539 entitled “COOLING DEVICE FOR REMOVING HEAT FROM SUBCUTANEOUS LIPID-RICH CELLS”;

U.S. Patent Publication No. 2008/0077201 entitled “COOLING DEVICES WITH FLEXIBLE SENSORS”;

U.S. Pat. No. 9,132,031 entitled “COOLING DEVICE HAVING A PLURALITY OF CONTROLLABLE COOLING ELEMENTS TO PROVIDE A PREDETERMINED COOLING PROFILE”;

U.S. Patent Publication No. 2009/0118722, filed Oct. 31, 2007, entitled “METHOD AND APPARATUS FOR COOLING SUBCUTANEOUS LIPID-RICH CELLS OR TISSUE”;

U.S. Patent Publication No. 2009/0018624 entitled “LIMITING USE OF DISPOSABLE SYSTEM PATIENT PROTECTION DEVICES”;

U.S. Pat. No. 8,523,927 entitled “SYSTEM FOR TREATING LIPID-RICH REGIONS”;

U.S. Patent Publication No. 2009/0018625 entitled “MANAGING SYSTEM TEMPERATURE TO REMOVE HEAT FROM LIPID-RICH REGIONS”;

U.S. Patent Publication No. 2009/0018627 entitled “SECURE SYSTEM FOR REMOVING HEAT FROM LIPID-RICH REGIONS”;

U.S. Patent Publication No. 2009/0018626 entitled “USER INTERFACES FOR A SYSTEM THAT REMOVES HEAT FROM LIPID-RICH REGIONS”;

U.S. Pat. No. 6,041,787 entitled “USE OF CRYOPROTECTIVE AGENT COMPOUNDS DURING CRYOSURGERY”;

U.S. Pat. No. 8,285,390 entitled “MONITORING THE COOLING OF SUBCUTANEOUS LIPID-RICH CELLS, SUCH AS THE COOLING OF ADIPOSE TISSUE”;

U.S. Provisional Patent Application Ser. No. 60/941,567 entitled “METHODS, APPARATUSES AND SYSTEMS FOR COOLING THE SKIN AND SUBCUTANEOUS TISSUE”;

U.S. Pat. No. 8,275,442 entitled “TREATMENT PLANNING SYSTEMS AND METHODS FOR BODY CONTOURING APPLICATIONS”;

U.S. patent application Ser. No. 12/275,002 entitled “APPARATUS WITH HYDROPHILIC RESERVOIRS FOR COOLING SUBCUTANEOUS LIPID-RICH CELLS”;

U.S. patent application Ser. No. 12/275,014 entitled “APPARATUS WITH HYDROPHOBIC FILTERS FOR REMOVING HEAT FROM SUBCUTANEOUS LIPID-RICH CELLS”;

U.S. Pat. No. 8,603,073 entitled “SYSTEMS AND METHODS WITH INTERRUPT/RESUME CAPABILITIES FOR COOLING SUBCUTANEOUS LIPID-RICH CELLS”;

U.S. Pat. No. 8,192,474 entitled “TISSUE TREATMENT METHODS”;

U.S. Pat. No. 8,702,774 entitled “DEVICE, SYSTEM AND METHOD FOR REMOVING HEAT FROM SUBCUTANEOUS LIPID-RICH CELLS”;

U.S. Pat. No. 8,676,338 entitled “COMBINED MODALITY TREATMENT SYSTEMS, METHODS AND APPARATUS FOR BODY CONTOURING APPLICATIONS”;

U.S. Pat. No. 9,314,368 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Pat. No. 9,844,461 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Publication No. 2012/0239123 entitled “DEVICES, APPLICATION SYSTEMS AND METHODS WITH LOCALIZED HEAT FLUX ZONES FOR REMOVING HEAT FROM SUBCUTANEOUS LIPID-RICH CELLS”;

U.S. Pat. No. 9,545,523 entitled “MULTI-MODALITY TREATMENT SYSTEMS, METHODS AND APPARATUS FOR ALTERING SUBCUTANEOUS LIPID-RICH TISSUE”;

U.S. Pat. No. 9,844,460 entitled “TREATMENT SYSTEMS WITH FLUID MIXING SYSTEMS AND FLUID-COOLED APPLICATORS AND METHODS OF USING THE SAME”;

U.S. Provisional Patent Application No. 61/943,251 entitled “TREATMENT SYSTEMS AND METHODS FOR TREATING CELLULITE”; and

U.S. Provisional Patent Application No. 61/943,257 entitled “TREATMENT SYSTEMS, METHODS, AND APPARATUS FOR REDUCING IRREGULARITIES CAUSED BY CELLULITE.”

### TECHNICAL FIELD

The present disclosure relates generally to treatment systems and methods for cooling targeted tissue. In particular, several embodiments are directed to treatment systems and methods for cooling tissue to improve the appearance of treatment sites with gynoid lipodystrophy or other skin irregularities. Embodiments are also disclosed for improving the appearance of skin, body contouring, and treating various skin conditions.

### BACKGROUND

It is often desirable to improve the appearance of bodies in many respects and treat various skin conditions. One example is the unattractive appearance of cellulite (gynoid lipodystrophy). Cellulite can be caused by subcutaneous fat lobules protruding or penetrating into the dermis and can be the consequence of, for example, an engorged adipose layer sequestered within the deep subcutis hypodermal fibrous septa, a weakened and/or degraded extracellular matrix, microcirculation compression resulting in decreased oxygen tension and hypoxia, and inflammatory edema. Cellulite is typically recognized by localized skin surface nodularity and dimpling considered to be cosmetically unappealing and often referred to as a cottage cheese appearance or an orange peel appearance. Unfortunately, cellulite has proved to be a difficult and vexing problem to treat, although the demand for an effective treatment has been and remains quite high.

Other examples where improvement of body appearance are needed are in the fields of body contouring and skin appearance. Improvements are also desired in treating various skin conditions, such as hyperhidrosis. Hyperhidrosis is a condition associated with excessive sweating that results from the overproduction and secretion of sweat from sweat glands and can cause discomfort and embarrassment.

Accordingly, it is an objective of various embodiments of the present invention to address these and other needs.

### BRIEF DESCRIPTION OF THE DRAWINGS

In the drawings, identical reference numbers identify similar elements or acts. The sizes and relative positions of elements in the drawings are not necessarily drawn to scale.

FIG. 1A is a schematic cross-sectional view of the skin, dermis, and subcutaneous tissue of a subject with cellulite.

FIG. 1B is a schematic cross-sectional view of the skin, dermis, and subcutaneous tissue of the subject in FIG. 1A showing a reduction in the appearance of cellulite. A treatment device is shown applied to the skin.

FIG. 2 is a partially schematic, isometric view of a treatment system for non-invasively removing heat from target areas of a subject in accordance with an embodiment of the technology.

FIGS. 3 to 7 are flow diagrams illustrating methods for treating target areas in accordance with embodiments of the technology.

FIG. 8 is a partially schematic, isometric view of a treatment system for non-invasively removing heat from target areas of a subject in accordance with an embodiment of the technology.

FIG. 9 is a partial cross-sectional view illustrating a treatment device suitable to be used in treatment systems in accordance with embodiments of the technology.

FIGS. 10A to 10C are schematic, cross-sectional views illustrating treatment devices in accordance with embodiments of the technology.

FIG. 11 is a partial cross-sectional view illustrating a vacuum treatment device in accordance with another embodiment of the technology.

FIG. 12 is a schematic block diagram illustrating computing system software modules and subcomponents of a computing device suitable to be used in treatment systems in accordance with embodiments of the technology.

### DETAILED DESCRIPTION

### A. Overview

The present disclosure describes treatment systems and methods for cooling tissue to produce freeze-induced injuries for improving the appearance of areas with gynoid lipodystrophy or other irregularities, improving the appearance of skin, body contouring, treating various skin conditions, or combinations thereof. Several of the details set forth below are provided to describe the following examples and methods in a manner sufficient to enable a person skilled in the relevant art to practice, make, and use them. Several of the details and advantages described below, however, may not be necessary to practice certain examples and methods of the technology. Additionally, the technology may include other examples and methods that are within the scope of the technology but are not described in detail.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, blocks, stages, or characteristics may be combined in any suitable manner in one or more examples of the technology. The headings provided herein are for convenience only and are not intended to limit or interpret the scope or meaning of the technology.

Various aspects of the technology are directed to treatment methods for affecting cellulite of a human subject's body and other treatments. In one embodiment, the method can include removing heat from a target region between the subject's epidermis and subdermal tissue to produce a freeze event localized in the dermal layer which causes a reduction of visible cellulite. In various embodiments, the target region can be cooled to a temperature equal to or less than about −1° C., −2° C., −5° C., −10° C., −15° C., −20° C., −30° C., or −40° C. at a depth equal to or greater than about 1 mm, 2 mm, 3 mm, or 4 mm. In some embodiments, most of the partially or totally frozen tissue by volume can be in a single layer of tissue, such as the epidermal layer, dermal layer, or subcutaneous layer. In other embodiments, multiple layers of tissue can be frozen. The subject's skin can be periodically or continuously heated to limit or prevent thermal damage to non-targeted tissue, in particular to sometimes protect the epidermal layer.

At least some embodiments are directed to reducing or eliminating cellulite, wrinkles, loose skin, sagging skin, poor skin tone or texture, and other skin irregularities often considered to be cosmetically unappealing. As used herein, “improving the appearance of cellulite” is intended to include any combination of improving skin tone, thickening of the skin, improving tissue elasticity, or other similar effects to reduce cellulite. As used herein, “improving the appearance of skin” is intended to include any combination of skin tightening, improving skin tone or texture, thickening of the skin, elimination or reducing fine lines and wrinkles or deeper wrinkles, increasing skin smoothness, or improving the appearance of cellulite, or other similar effects. What is not included in these terms is treating the skin to such an extent so as to cause hyperpigmentation (skin darkening) and/or hypopigmentation (skin lightening) either immediately after the treatment or hours or a day or days or weeks thereafter.

Some aspects of the technology are directed to treatment methods for affecting tissue of a human subject's body. In one embodiment, the method can include cooling tissue to produce a freeze event that reduces or eliminates cellulite by affecting at least one of skin tone, thicknesses of the tissue layers (e.g., increasing the thicknesses of the dermal layer and/or epidermal layer), and/or tissue elasticity. In certain embodiments, the method also includes inhibiting damage to non-targeted tissue of the subject's skin while producing the freeze event. In some embodiments, the freeze event can include injury to at least some of the subject's skin (e.g., epidermis, dermis, etc.), subcutaneous adipose tissue, or other targeted tissue.

Freeze events can result in freeze trauma and/or freeze injury that can be contained in a layer of tissue. In embodiments in which the freeze event is centralized in the dermis, the treatment method can include inhibiting damage to the epidermis while producing the freeze event. A cryoprotectant can be applied to the surface of the subject's skin prior to or during heat removal to protect an epidermis and possibly other layers. In other embodiments, energy (e.g., heat, radiofrequency energy, electromagnetic energy, electric fields, ultrasound energy, light energy, etc.) can be applied to the subject's skin to inhibit damage to the epidermis. Excessive damage to the epidermis can sometimes lead to undesired skin coloration issues. In some supercooling embodiments, supercooling can be used to target tissue at the desired depth without using any cryoprotectant. Non-targeted tissue in the skin, such as the epidermis, can be heated above its freezing point before initiating crystallization of the remaining supercooled tissue. Accordingly, crystallization in the supercooled tissue can be induced without damaging the subject's epidermis and possibly some deeper skin layers. In some embodiments, the surface of the skin and tissue therebeneath is supercooled. The surface of the skin is then warmed above a freezing temperature. After warming the surface of the skin, the supercooled tissue is nucleated to initiate a partial or total freeze event.

With or without freezing, at least some embodiments of the technology are directed to controlling a cooling device or providing other means for sufficiently protecting the epidermis from injuries that cause hyperpigmentation (skin darkening) and/or hypopigmentation (skin lightening). The other means for protection can include, without limitation, heating the epidermis to a non-freezing temperature while deeper tissue remains cold to induce injury thereto and/or applying a cryoprotectant to a surface of the skin to provide freeze protection to the epidermis while allowing deeper tissue to be more affected by the cooling/cold treatment.

At least some embodiments of non-invasive treatments for reducing cellulite can include producing wound remodeling (e.g., healing) phase resulting from freeze-induced tissue trauma to produce enhancements in structural integrity of the epidermal-dermal junction and/or epidermal textural quality. The freeze-induced trauma can include thermal injuries that are selected to reduce skin surface irregularities caused by cellulite.

Further aspects of the technology are directed to systems and methods for affecting a target region of a human subject's body by removing heat from the target region to alter subdermal connective tissue while lipid-rich cells in the subcutaneous layer are not substantially affected. The method can optionally include applying a cryoprotectant and/or warming the surface of the skin. For example, in one embodiment, heat removal includes freezing tissue of the fibrous septum to affect the fibrous septum while the epidermis is not substantially affected. The thermal injury can cause beneficially generation of fibrous tissue, remodeling of the fibrous septum, or the like. In various embodiments, heat can be removed from the target region to affect cells (e.g., reduce, destroy, and/or damage cells), alter tissue characteristics, or combinations thereof. In some embodiments, a treatment system can (1) reduce fat in the superficial compartment, (2) alter skin tone/thickness/elasticity due to a freeze event (e.g., cold injury) and resultant healing cascade, and/or (3) affect connective tissue (including the septa) as a result of cold injury and resultant healing.

Tissue characteristics affected by cryotherapy can include, without limitation, tissue strength, tissue elasticity, tissue layer thickness, and/or skin tone. In some embodiments, cryotherapy can increase the elasticity of a targeted tissue layer, such as the skin, epidermis, and/or dermis. For example, the dermis can be cooled to produce a freeze event that causes thermal injury to the dermal tissue. The resultant wound remodeling can increase the strength and/or elasticity of the dermis. The severity of the freeze event can be selected to achieve the desired change in strength and/or elasticity while non-targeted tissue, such as epidermal or subdermal tissue, can remain generally unaffected. In some embodiments, cryotherapy can increase the thicknesses of multiple layers of tissue. For example, the epidermis and dermis can be wounded to increase their thicknesses due to wound remodeling. In yet further embodiments, freeze injury causes fibrosis in the form of fibroblast proliferation and/or increased collagen.

At least some freeze events disclosed herein can promote a natural body response that reduces an orange peel appearance, a cottage cheese appearance, etc. The subject's natural body response can include thickening of the epidermis, dermis, layers of connective tissue (e.g., regions of cellular matrix overlaying fat pockets), and/or other subdermal tissues. Layers for thickening can be selected based on the ability to control the freeze events and the location and extent of the associated injury/trauma. For example, one or more layers of the epidermis (e.g., stratum corneum, stratum lucidum, stratum granlosum, stratum spinosum, and/or stratum basale) can be the site of wound formation and remodeling. The papillary layer and/or reticular layer of the dermis can also be the site of wound formation and remodeling.

At least some embodiments are systems and methods for selective non-invasive cooling of tissue to produce a freeze event at a depth equal to or greater than about 1 mm, 2 mm, 3 mm, or 5 mm. The depth can be selected based on the location of the treatment site (e.g., hips, buttock, thighs, etc.). The target region can be cooled to a temperature equal to or lower than about 0° C., −5° C., −10° C., −15° C., −20° C., −25° C., −30° C., −35° C., or −40° C. for a treatment period. The treatment period can be equal to or longer than about 1 second, 2 seconds, 3 seconds, 5 seconds, 30 seconds, 1 minute, 5 minutes, 10 minutes, 30 minutes, or other time periods selected based on the location of targeted tissue and/or desired resultant healing.

Applicators disclosed herein can include elements (e.g., electrodes, vibrators, etc.) for delivering energy, such as radiofrequency energy, electromagnetic energy, infrared energy, light energy, ultraviolet energy, microwave energy, ultrasound energy (e.g., low frequency ultrasound, high frequency ultrasound, etc.), mechanical massage, and/or electric fields (e.g., AC or DC electric fields). The energy can inhibit or reduce freeze damage in non-targeted regions, such as an epidermis, while allowing more aggressive cooling of a targeted region. In non-targeted cells or structures, non-thermal energy parameters may be selected to reduce ice crystal nucleation, size and/or length, reduce freezing lethality, or the like. In targeted cells or structures, non-thermal energy may be used to initiate crystal nucleation, growth, etc. Thus, non-thermal energy can be selectively applied to control cryotherapy. Thermal energy can be used to protect non-targeted tissue, such as facial subcutaneous fat, when cryogenically treating superficial dermal structures and/or epidermal structures on the face. Additionally or alternatively, non-targeted tissue can be protected by a chemical cryoprotectant. Some applicators can treat epidermal and/or dermal structures, such as collagen and/or elastin for skin tightening and dermal thickening, glands (e.g., apocrine glands, eccrine glands, etc.), nerve tissue (e.g., superficial nerves), and/or hair follicles.

At least some aspects are directed to systems and devices that enable supercooling of target tissue to alter and reduce adipose tissue, contour sites, or perform other cryotherapy procedures. Aspects of the disclosure are further directed to systems or methods for protecting non-targeted cells, such as non-lipid-rich cells (e.g., cells in the dermal and/or epidermal skin layers), by preventing or limiting freeze damage during dermatological and related aesthetic procedures that require sustained exposure to cold temperatures. For example, treatment systems and devices can supercool treatment sites without nucleating crystals. Non-targeted tissue can be heated to localize the supercooling, and after localizing the supercooled tissue, supercooled body fluids/lipids can then be intentionally nucleated to damage, reduce, disrupt, or otherwise affect targeted cells. Nucleation can be induced by delivering an alternating current to the tissue, applying a nucleating solution onto the surface of the skin (for example one that includes bacteria which initiate nucleation), and/or by creating a mechanical perturbation to the tissue, such as by use of vibration, ultrasound energy, etc.

### B. Cellulite

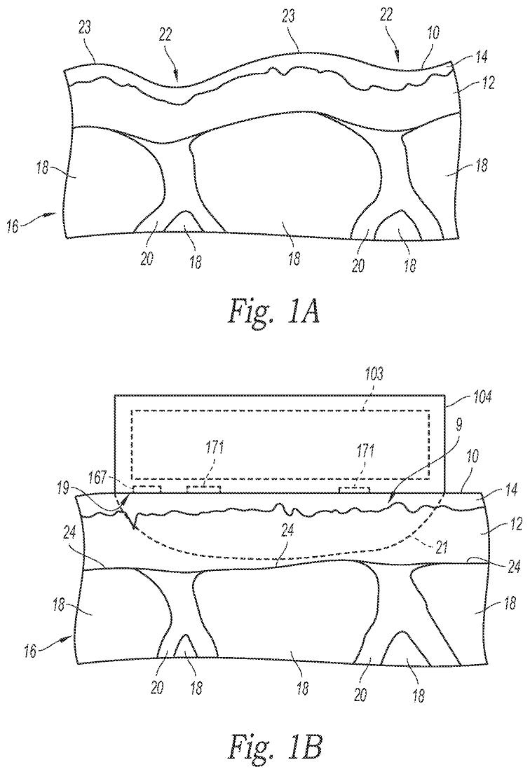

FIG. 1A is a schematic cross-sectional view of tissue with gynoid lipodystrophy. Gynoid lipodystrophy typically is a hormonally mediated condition characterized by the uneven distribution of adipose tissue in the subcutaneous layer that gives rise to an irregular, dimpled skin surface common in women. Cellulite-prone tissue can be characterized by the uneven thickness and distribution of some fibrous septae strands. Piérard, G. E., Nizet, J. L, Piérard-Franchimont, C., “Cellulite: From Standing Fat Herniation to Hypodermal Stretch Marks,” Am. J. Dermatol. 22:1, 34-37 (2000). As shown schematically, cottage-cheese like dimpling of the skin 10 may be located, for example, along the legs (e.g., thighs, buttock, etc.). The dermis 12 is between the epidermis 14 and subcutaneous layer 16. The subcutaneous layer 16 has connective collagenous tissue called fibrous septae 20 that subdivides adipose tissue into fat cell chambers or lobules 18 (also called “papillae adiposae”). The fibrous septae 20, which for females tend to generally be oriented perpendicular to the skin surface and anchor the dermal layers 12 to the underlying fascia and muscle (not shown), are organized within the subcutaneous layer 16 to form a connective web around the fat lobules 18. Subcutaneous adipose cells and their lobules 18 are not uniformly distributed throughout the subcutaneous tissue layer 16 and exhibit regional differences in size and shape. These regional differences can, in part, be due to gender, age, genetics, hormones and physical conditioning among other physiological factors.

The number, sizes, distribution, and orientation of the fibrous septae 20 also vary by body location, gender, and age. Histological studies have shown that fibrous septae architecture in females differs from that in males. In males, fibrous septae 20 tend to form an intersecting network that divide the papillae adiposae into small, polygonal units. In contrast, fibrous septae 20 in females tend to be oriented perpendicularly to the cutaneous surface, creating fat cell chambers that are columnar in shape and sequestered by the connective strands and the overlaying dermis layer 12. When the intersecting fibrous septae 20 are more uniform in size and elasticity as is often a characteristic of males, the forces within and between the fibrous septae and their surrounding tissue tend to be distributed relatively evenly. However, the columnar architecture of the fibrous septae 20 found in some females can result in an uneven distribution of forces throughout the subcutaneous tissue. In particular, and without being bound by theory, it is believed that this uneven distribution of forces is partially manifested by the columnar fibrous septae 20 being held in a state of tension by the underlying fascia and other tissue, resulting in a tethering or anchoring effect at the point where each such septum 20 connects with the dermal tissue 12. This tethering or anchoring is in turn manifested at the skin surface as low spots 22. The septae tends to herniate as the papillae adiposae 18 bulge into the dermal tissue 12 and result in high spots 23. When viewed over a larger area of a few square centimeters, the non-homogeneous nature of the skin surface's relative high and low spots results in a dimpled or irregular appearance characteristic of cellulite often referred to as a cottage cheese appearance or orange peel appearance.

The vertical pull of the fibrous septum 20 (e.g., pulling in a direction substantially perpendicular to the skin) can be reduced eliminate or reduce bumpiness of the subdermal fat lobules 18. Wound remodeling affect the tension in connective tissue extending from the dermis 12 to the subcutaneous muscle (not shown). Localized freezing of the subcutaneous tissue 16 can reduce the number and/or size of lipid-rich cells of the fat lobules 18 to reduce lobule bulging. Additionally or alternatively, vertical bands of the fibrous septum 20, or portions of the bands, to reduce pulling on the dermis 12, thus reducing bulging of fat lobules 18. In some embodiments, the cellular matrix between the fat lobules 18 and the dermis 12 can be damaged in order to produce remodeling that ultimately increases the strength and/or elasticity of the cellular matrix. Freezing of dermal tissue can produce wound healing/remodeling that strengthens and/or thickens the dermal tissue to help flatten the fat lobules. Freezing of epidermal tissue can produce wound healing/remodeling that strengthens and/or thickens the epidermal tissue to further flatten the skin 10. Accordingly, cryotherapy can be designed to target the epidermis, dermis, and/or subcutaneous structures.

FIG. 1B is a schematic cross-sectional view of the skin 10 and subcutaneous adipose tissue 16 of the subject in FIG. 1A showing a reduction in the appearance of cellulite in accordance with aspects of the present technology. A treatment device in the form of a thermoelectric applicator 104 (“applicator 104”) has been applied to and cooled the skin 10 to produce a freeze-induced injury that affected the epidermis 14, dermis 12, fibrous septae 20, and/or subcutaneous adipose tissue 16 to minimize, reduce, or eliminate at least the appearance of gynoid lipodystrophy. FIG. 1B shows the skin 10 after cryotherapy has been performed. The interface between the lobules 18 and the dermis 12 can be generally flat to help flatten the surface of skin 10. As shown in FIGS. 1A and 1B, the lobules 18 have flattened so that lobule flat regions 24 face the dermis 12. As such, the treated skin 10 of FIG. 1B can have a much more even or regular/smooth appearance than the skin 10 of FIG. 1A.

### C. Cryotherapy

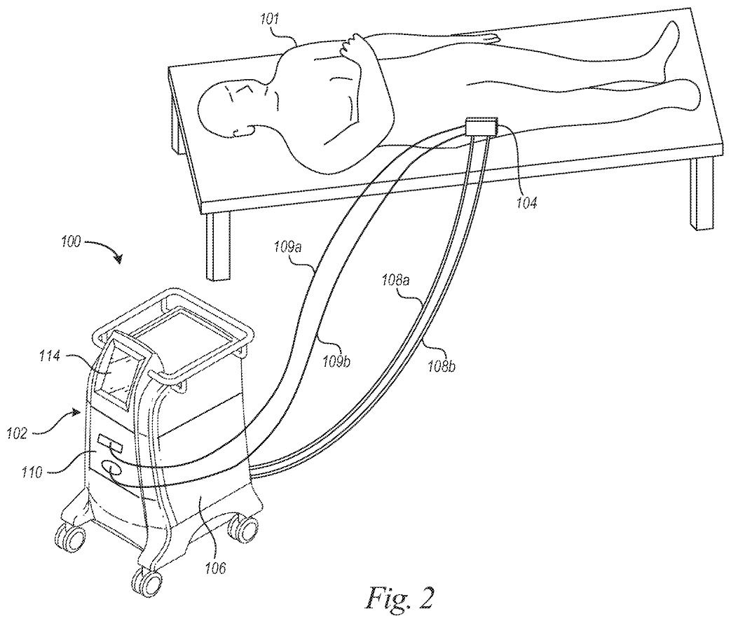

FIG. 2 and the following discussion provide a brief, general description of an example of a suitable treatment system 100 in which aspects of the technology can be implemented. The treatment system 100 can be configured to control hypothermia to treat the site of cellulite manifested by skin dimpling and nodularity. In some cellulite treatments, the treatment system 100 can weaken, destroy, or otherwise injure the tissue (e.g., fibrous septum or other connective tissue) that pulls on the dermis. It is also thought that the wound-healing response following a freeze-induced injury caused by the applicator 104 can result in remodeling of the underlying connective tissue and changes in skin characteristics (e.g., increased in thickness) that otherwise reduce or alter the appearance of cellulite. The freeze wound can result in tissue damage and/or destruction of the cells and connective tissue with reference to the superficial skin layers (e.g., epidermis and/or dermis). The dedicated wound repair and the wound remodeling process can result in the enhancement of the structural integrity and textural quality of skin and, thereby, restore the skin to a desired clinical outcome. The wound remodeling process can be selected based on the damage potential, repair process, or the like. The mechanisms of tissue injury in cryotherapy can involve direct cellular injury (e.g., damage to the cellular machinery), vascular injury, and/or freeze-stimulated immunologic injury.

The system 100 can also freeze the upper layers of skin. Without being bound by theory, it is believed that low temperatures may potentially cause damage in the epidermis and/or dermis via at least intracellular and/or extracellular ice formation. The ice may expand and rupture the cell wall, but it may also form sharp crystals that locally pierce the cell wall as well as vital internal organelles, either or both resulting in cell death. When extracellular water freezes to form ice, the remaining extracellular fluid becomes progressively more concentrated with solutes. The high solute concentration of the extracellular fluid may cause intracellular fluid be driven through the semi-permeable cellular wall by osmosis resulting in cell dehydration and death. Such selective tissue injury can induce healing events in the tissue that has positive effects on skin appearance including a reduction in surface irregularities in the surface of the skin.

As shown in FIG. 1B, the applicator 104 has a heat-exchanging surface 19 in thermal contact with a treatment site 9 along the skin 10. A heating cooling device 103 of the applicator 104 can cool and affect tissue at a cooling zone 21 (shown in phantom line) with dimensions and shape selected based on the cryotherapy procedure to be performed. A central region of the cooling zone 21 can be at a maximum depth of, for example, about 0.25 mm to about 2 mm, about 0.25 mm to about 1 mm, about 0.5 mm to about 1 mm, about 0.5 mm to about 2 mm, or about 0.5 mm to about 4 mm. In some embodiments, the cooling zone 21 can comprise mostly epidermal and dermal tissue. Surrounding tissue may also be cooled but can be at sufficiently high temperatures to avoid thermal injury. In some procedures, the cooling zone 21 can comprise most of the tissue located directly between the heat-exchanging surface 19 and a region of the subcutaneous tissue 16 directly beneath the heat-exchanging surface 19. For example, at least 60%, 70%, 80%, 90%, or 95% of the tissue directly between the heat-exchanging surface 19 and the subcutaneous tissue can be located within the cooling zone 21. In other procedures, the cooling zone 21 can be deep enough to include subcutaneous tissue 16 to target lipid-rich cells of the lobules 18, the fibrous septum 20, or other subcutaneous tissue. The temperature profile across the heat-exchanging surface 19 can be constant or varying to achieve the desired cooling zone 21.

The applicator 104 of FIGS. 1B and 2 can also thermally damage subdermal tissue, such as the connective tissue of the fibrous septum (i.e., bands of connective tissue extending generally perpendicular to the skin) to help lengthen the connective tissue, thereby reducing bulging of the fat lobules toward the dermis. Additionally, the connective tissue can be altered without substantially affecting other tissue. In some procedures, the connective tissue of the fibrous septum is affected while lipid-rich cells in the subcutaneous adipose tissue are not substantially affected by controlling the cooling to not sufficiently cool the deeper subcutaneous adipose tissue to inflict injury thereto. In other procedures, the connective tissue and the lipid-rich cells in the subcutaneous layer (e.g., subcutaneous adipose tissue) are affected in the same cooling routine or sequentially performed cooling routines while epidermal tissue and/or dermal tissue are not substantially affected by either heating these latter layers or using a cryoprotectant to protect them. The applicator 104 can also be suitable for reducing skin surface irregularities, such as dimpling and nodularity usually associated with cellulite, by cooling or freezing cells residing in the superficial skin layers (e.g., dermal and epidermal layers). Tissue alteration by cooling and/or freezing is believed to be an intermediate and/or final result of one or more mechanisms acting alone or in combination. It is thought that a wound-healing response following a freeze-induced injury can result in remodeling of the underlying connective tissue and changes skin characteristics (e.g., increased in skin thickness) that otherwise reduce or alter the appearance of cellulite.

In several embodiments, apoptosis of the subcutaneous lipid-rich cells is a desirable outcome for beneficially altering (e.g., sculpting and/or reducing) adipose tissue contributing to cellulite. Apoptosis, also referred to as “programmed cell death”, is a genetically-induced death mechanism by which cells self-destruct without incurring damage to surrounding tissues. An ordered series of biochemical events may induce cells to morphologically change. These changes include cellular blebbing, loss of cell membrane asymmetry and attachment, cell shrinkage, chromatin condensation, and chromosomal DNA fragmentation. Injury via an external stimulus, such as cold exposure, is one mechanism that can induce apoptosis in cells. Nagle, W. A., Soloff, B. L., Moss, A. J. Jr., Henle, K. J. “Cultured Chinese Hamster Cells Undergo Apoptosis After Exposure to Cold but Nonfreezing Temperatures” Cryobiology 27, 439-451 (1990). One aspect of apoptosis, in contrast to cellular necrosis (a traumatic form of cell death causing, and sometimes induced by, local inflammation), is that apoptotic cells express and display phagocytic markers on the surface of the cell membrane, thus marking the cells for phagocytosis by, for example, macrophages. As a result, phagocytes can engulf and remove the dying cells (e.g., the lipid-rich cells) without eliciting an immune response.

Without being bound by theory, one mechanism of apoptotic lipid-rich cell death by cooling is believed to involve localized crystallization of lipids within the adipocytes or other lipid-producing cells (e.g., residing in exocrine cells) at temperatures that do not induce crystallization in non-lipid-rich cells. The crystallized lipids may selectively injure these cells, inducing apoptosis (and may also induce necrotic death if the crystallized lipids damage or rupture the bilayer lipid membrane of the adipocyte). Another mechanism of injury involves the lipid phase transition of those lipids within the cell's bilayer lipid membrane, which results in membrane disruption, thereby inducing apoptosis. This mechanism is well documented for many cell types and may be active when adipocytes, or lipid-rich cells, are cooled. Mazur, P., “Cryobiology: the Freezing of Biological Systems” Science, 68: 939-949 (1970); Quinn, P. J., “A Lipid Phase Separation Model of Low Temperature Damage to Biological Membranes” Cryobiology, 22: 128-147 (1985); Rubinsky, B., “Principles of Low Temperature Preservation” Heart Failure Reviews, 8, 277-284 (2003). Other possible mechanisms of adipocyte damage, described in U.S. Pat. No. 8,192,474, relates to ischemia/reperfusion injury that may occur under certain conditions when such cells are cooled as described herein. For instance, during treatment by cooling as described herein, the targeted adipose tissue (e.g., lipid-rich cells in the lobules 18 of FIG. 1A) may experience a restriction in blood supply and thus be starved of oxygen due to isolation while pulled into, e.g., a vacuum cup, or simply as a result of the cooling which may affect vasoconstriction in the cooled tissue. In addition to the ischemic damage caused by oxygen starvation and the build-up of metabolic waste products in the tissue during the period of restricted blood flow, restoration of blood flow after cooling treatment may additionally produce reperfusion injury to the adipocytes due to inflammation and oxidative damage that is known to occur when oxygenated blood is restored to tissue that has undergone a period of ischemia. This type of injury may be accelerated by exposing the adipocytes to an energy source (via, e.g., thermal, electrical, chemical, mechanical, acoustic or other means) or otherwise increasing the blood flow rate in connection with or after cooling treatment as described herein. Increasing vasoconstriction in such adipose tissue by, e.g., various mechanical means (e.g., application of pressure or massage), chemical means or certain cooling conditions, as well as the local introduction of oxygen radical-forming compounds to stimulate inflammation and/or leukocyte activity in adipose tissue may also contribute to accelerating injury to such cells. Other yet-to-be understood mechanisms of injury may also exist.

In addition to the apoptotic mechanisms involved in lipid-rich cell death, local cold exposure may induce lipolysis (i.e., fat metabolism) of lipid-rich cells. For example, cold stress has been shown to enhance rates of lipolysis from that observed under normal conditions which serves to further increase the volumetric reduction of subcutaneous lipid-rich cells. Vallerand, A. L., Zamecnik. J., Jones, P. J. H., Jacobs, I. “Cold Stress Increases Lipolysis, FFA Ra and TG/FFA Cycling in Humans” Aviation, Space and Environmental Medicine 70, 42-50 (1999).

Without being bound by theory, the selective effect of cooling on lipid-rich cells is believed to result in, for example, membrane disruption, shrinkage, disabling, destroying, removing, killing, or another method of lipid-rich cell alteration. For example, when cooling the subcutaneous tissues to a temperature lower than 37° C., subcutaneous lipid-rich cells can selectively be affected. In general, the cells in the epidermis and dermis of the subject 101 have lower amounts of lipids compared to the underlying lipid-rich cells forming the subcutaneous tissues. Since lipid-rich cells are more sensitive to cold-induced damage than non-lipid-rich epidermal or dermal cells, it is possible to use non-invasive or minimally invasive cooling to destroy lipid-rich cells without harming the overlying skin cells. In one embodiment, thermal conduction can be used to cool the desired layers of skin to a temperature above the freezing point of water, but below the freezing point of fat to reduce the number and/or size of lipid-rich lobules in the subcutaneous layer at a target region.

In a typical procedure, a treatment device is positioned at least proximate to the surface of a subject's skin and heat is removed from the underlying tissue through the upper layers of the skin. This creates a thermal gradient with the coldest temperatures in the uppermost layers of skin near the cooling element. When cooling subcutaneous lipid-rich cells, the resulting thermal gradient causes the temperature of the upper layer(s) of the skin to be lower than that of the targeted underlying lipid-rich cells. For example, the treatment system 100 can cool the surface of the skin to about −20° C. to about 20° C. In other embodiments, the skin temperature can be from about −40° C. to about 10° C., from about −20° C. to about 10° C., from about −18° C. to about 5° C., from about −15° C. to about 5° C., or from about −15° C. to about 0° C. In further embodiments, the surface of the skin can be cooled to lower than about −10° C., or in yet another embodiment, lower than about −15° C. to about −25° C., −30° C., −35° C., or −40° C. In further embodiments, the skin temperature can be lower than −25° C. to induce a deep freeze wound.

### 1. Tissue Injuries

Mechanisms of tissue injury in cryotherapy can involve direct cellular injury (e.g., damage to the cellular machinery) and/or vascular injury. For example, cellular injury can be controlled by thermal parameters, including (1) cooling rate, (2) end (or minimum) temperature, (3) time held at the minimum temperature (or hold time), and (4) thawing rate. In one example, increasing the hold time (e.g., at the minimum temperature) can allow the intracellular compartments to equilibrate with the extracellular space, thereby increasing cellular dehydration. Likewise, freeze events can also destroy or injure the microvasculature, the site of nutrient and oxygen delivery, thus causing necrosis in some examples. A common source for vascular injury is damage to the vessel wall due to vessel distension and engorgement from perivascular cellular dehydration. Additionally, vascular tissue injury can occur during tissue thawing. For example, high oxygen delivery to the tissue that occurs with hyperperfusion may cause free radical formation, which can, in turn, cause endothelial damage. In some embodiments, administration of free radical inhibitors may be able to limit this form of endothelial damage.

Another mechanism of freezing injury is freeze-stimulated immunologic injury. Without being bound by theory, it is believed that after cryotherapy, the immune system of the host is sensitized to the disrupted tissue (e.g., lethally damaged tissue, undamaged tissue or sublethally injured tissue), which can be subsequently destroyed by the immune system.

### 2. Freeze Events

Freeze events can elicit a desired response to minimize, reduce, or eliminate the appearance of cellulite. The freeze event can produce enhancements in structural integrity of target regions (e.g., the epidermal-dermal junction) and epidermal textural quality in the non-invasive treatment of cellulite. The location and extent of the crystallization can be selected based on the desired effects to the skin, epidermis, stratum corneum, or other targeted or non-targeted tissue.

One cryotherapy procedure involves at least partially or totally freezing tissue to form crystals that alter targeted cells to cause skin tightening, skin thickening, fibrosis, etc. without destroying a significant amount of cells in the skin. The surface of the patient's skin can be cooled to temperatures no lower than, for example, −40° C. for a duration short enough to avoid, for example, excessive ice formation, permanent thermal damage, or significant hyperpigmentation or hypopigmentation. In another embodiment, destruction of skin cells can be avoided by periodically or continually applying heat to the surface of the patient's skin to keep or raise the skin's temperature above a freezing temperature. For example, the skin can be warmed to a temperature greater than 0° C., greater than 10° C., greater than 20° C., greater than 30° C., or other temperature sufficient to avoid, for example, excessive ice formation, permanent thermal damage, or significant hyperpigmentation or hypopigmentation of the non-targeted and/or epidermal tissue. In some treatments, the surface of the skin can be cooled to produce partial or total freeze events that cause apoptotic damage to skin tissue without causing significant damage to adjacent subcutaneous tissue.

In some tissue-freezing procedures, the applicator 104 can controllably freeze tissue and can detect the freezing event. After detecting the freeze event, the applicator 104 can periodically or continuously remove heat from the target tissue to keep a volume of target tissue frozen for a suitable length of time to elicit a desired response. The detected freeze event can be a partial freeze event, a complete freeze event, etc. The freezing process can include forming crystals in intracellular and/or extracellular fluids (including lipids), and the crystals can be small enough to avoid disrupting membranes. This can prevent significant permanent tissue damage, such as necrosis. Some partial freeze events can include freezing mostly extracellular material without freezing a substantial amount of intercellular material, but other partial freeze events can include freezing mostly intercellular material without freezing a substantial amount of extracellular material.

The targeted tissue can remain in the frozen state long enough to be affected but short enough to avoid damaging non-targeted tissue. For example, the duration of the freeze event can be equal to, longer than, or shorter than about 10 seconds, 20 seconds, 30 seconds, or 45 seconds or about 1, 2, 3, 4, 5, or 10 minutes. The frozen tissue can be thawed to prevent necrosis and, in some embodiments, can be thawed within a period of time (e.g., about 20 seconds, about 30 seconds, about 45 seconds, about 1 minute, about 2 minutes, about 3 minutes, about 4 minutes, about 5 minutes, or about 10 minutes) after initiation of the freeze event. In some embodiments, the controlled freezing causes tightening of the skin, thickening of the skin, and/or a cold shock response at the cellular level in the skin. In one tissue-freezing embodiment, the applicator 104 can produce a freeze event that includes, without limitation, partial or full thickness freezing (e.g., partial or complete freezing) of the patient's skin for a relatively short period of time to avoid cooling the adjacent subcutaneous tissue to a low enough temperature for subcutaneous cell death. A freeze event in the form of freeze injury of short duration and of mild to moderate intensity may produce, for example, a thicker, more resilient epidermis, which may improve the surface textural quality. In one embodiment, the duration of the freeze event and temperature of the tissue can be selected to achieve a desired thickened resilient epidermis. The greater the duration of freezing, the deeper the penetration of the trauma and tissue destruction.

The application of simultaneous or successive light to moderate freeze trauma to skin overlying sites of cellulite, in conjunction with selective cryotherapy induced reduction of subcutaneous lipid-rich cells at the sites of cellulite, can drive the wound remodeling phase in producing desirable structural and textural enhancements to the unsightly dimpling and nodularity usually associated with this condition. Selective localized freeze trauma (e.g., with pre-selected exposure parameters) should not initially disturb the dermal tensile strength but a transient increase in ECM structural integrity associated with collagen synthesis can occur as the result of wound remodeling. The combination of a structurally competent ECM with a consequential thickening of the epidermal barrier can provide a desired clinical outcome.

### 3. Supercooling

A freezing point of a material is most reliably ascertained by warming frozen material slowly and measuring a temperature at which melting begins to occur. This temperature is generally not ambiguous if the material is slowly warmed. Partial melting will begin to occur at the freezing/melting point. Conversely, if a non-frozen material is cooled, its freezing/melting point is harder to ascertain since it is known that many materials can simply “supercool,” that is they can be cooled to a bulk temperature below their freezing/melting point and still remain in a non-frozen state. As used herein, “supercooling,” “supercooled,” “supercool,” etc., refers to a condition in which a material is at a temperature below its freezing/melting point but is still in an unfrozen or mostly unfrozen state.

If skin is cooled in a controlled manner, targeted tissues can generally be supercooled below their freezing points without forming nucleation sites and/or microscopic crystals in extracellular fluid and/or intracellular fluid and thereby such tissues can reside in a supercooled unfrozen state. After supercooling, the supercooled tissue can then be nucleated via a mechanical perturbation (e.g., vibration, ultrasound pulse, change in pressure, etc.) to at least partially freeze that tissue. In one embodiment, the mechanical perturbation can induce crystallization to produce a freeze event (e.g., a partial freeze event, a complete freeze event, etc.) that causes targeted cells to be destroyed or damaged by ice crystal formation in intracellular and/or extracellular fluids. Other nucleation methods can also be used, such as applying a solution with a nucleating agent (such as a nucleating bacteria) onto the skin, or by applying an electrical alternating current, RF energy, microwave energy, ultrasound energy, etc. to the skin.

The freezing process can include forming ice crystals small enough to avoid disrupting membranes to prevent significant permanent tissue damage (e.g., necrosis) but large enough to affect targeted cells. Some partial freeze events can include freezing mostly extracellular material without freezing a substantial amount of intercellular material. In other embodiments, partial freeze events can include freezing mostly intercellular material without freezing a substantial amount of extracellular material. Chemical cryoprotectants can be used to inhibit unwanted freezing of extracellular and intercellular material. In yet other embodiments, the partial freeze event can include freezing extracellular and intercellular material, and in other embodiments the material can be totally frozen. The frozen targeted tissue can remain in the frozen state long enough to be affected but short enough to avoid undesired thermal damage, including necrosis and/or damage to non-targeted cells. For example, the duration of the freeze event (e.g., the partial or complete freeze event) can be shorter or longer than about 10 seconds, 20 seconds, 30 seconds, or 45 seconds or about 1, 2, 3, 4, 5 or 10 minutes. The frozen tissue can be thawed to prevent undesired thermal damage and, in some embodiments, can be thawed within about 5 seconds, 10 seconds, 20 seconds, 30 seconds, or 45 seconds or about 1, 2, 3, 4, 5, or 10 minutes after initiation of the freeze event. In many embodiments, and as described in further detail herein, non-targeted cells can be protected by a warming cycle that brings the temperature of non-targeted cells to a temperature above their freezing temperatures prior to catalyzing a freeze event in the supercooled target tissue. For example, non-targeted tissue can be warmed to temperatures above about −1.8° C., above about 0° ° C., above about 5° C., above about 10° C., above about 20° C., above about 30° C., or above about 32° C. Warming can be accomplished by thermal heaters disposed on a surface of the applicator contacting or confronting a skin surface. Alternatively, if deeper tissue is not targeted, such tissue could be warmed using focused electrical currents which focus their energy below the skin surface, focused ultrasound which has a focal point for its energy below the skin surface, or RF energy.

As discussed above, deep hypodermal fat cells are more easily damaged by low temperatures than the overlying dermal and/or epidermal layers of skin, and, as such, thermal conduction can be used to cool the desired layers of skin to a supercooled temperature suitable to freeze lipid-containing cells upon perturbation (e.g., a nucleating event). However, there is an associated risk of also freezing the uppermost layers of skin. Without being bound by theory, it is believed that low temperatures may potentially cause damage in the epidermis (e.g., stratum corneum, stratum lucidum, stratum granulosum, stratum spinosum, stratum basale, etc.) via at least intracellular and/or extracellular ice formation. The ice may expand and rupture the cell wall, but it may also form sharp crystals that locally pierce the cell wall and vital internal organelles, either or both resulting in cell death. When extracellular liquid, such as water, freezes to form ice, the remaining extracellular fluid becomes progressively more concentrated with solutes. The high solute concentration of the extracellular fluid may cause intracellular fluid be driven through the semi-permeable cellular wall by osmosis resulting in cell dehydration and death. Accordingly, in one embodiment, mechanical perturbation and/or other catalyst for nucleation (e.g., RF energy, alternating electric fields, ultrasound energy, etc.) within the target tissue can be provided only following a protective increase of a temperature of non-targeted epidermal layers and/or dermal layers. The non-targeted layers can be warmed enough to avoid freezing of non-targeted tissue upon nucleation.

As explained in more detail below, the treatment systems disclosed herein can employ a temperature treatment cycle to (a) cool (e.g., supercool) target tissue, for example, to a temperature below freezing without causing nucleation or microscopic crystallization of intracellular and/or extracellular fluids and (b) warm non-targeted tissue to increase its temperature above its freezing temperature. After warming the non-targeted tissue, the treatment systems can induce nucleation and hence freezing in the supercooled target tissue. In certain embodiments, the treatment system 100 of FIG. 2 can supercool a volume of tissue and can warm superficial skin layers to prevent injury to those superficial skin layers without the use of a chemical cryoprotectant. Alternatively, a cryoprotectant can further be employed to provide an added element of safety to minimize chances that undesired skin layers are undesirably damaged, particularly epidermal tissue, so as to prevent or minimize any chance of creating hyperpigmentation or hypopigmentation.

Formation of nucleation sites can be catalyzed by perturbation of the supercooled tissue. In particular embodiments, the supercooled region (e.g., body fluids within the targeted tissue cooled below their freezing temperatures) can be subjected to vibrations, changes in mechanical pressure, and/or ultrasound pulse(s) provided by the applicator 104 to catalyze nucleation of the supercooled extracellular and/or intracellular fluids, lipids, etc. Nucleation perturbations can also be created by applying a nucleating solution to the skin, or by using electrical energy. The extracellular and/or intracellular fluids, lipids, etc. in the non-targeted skin layers under the applicator 104 can be conductively warmed during the treatment (e.g., following transdermal cooling of targeted tissue) such that freeze injury is avoided in the non-targeted tissue when nucleation is initiated.

In one embodiment, to achieve supercooled temperatures of the targeted tissue without initiating nucleation, the treatment site can be cooled at a relatively slow rate (e.g., the temperature profile can cause a slow cooling of the tissue at the target region). For example, the rate of cooling can be either equal to, slower or faster than about 0.5, 1, 2, 3, 4, 5, 6, 7, 8, 9 or 10 degrees C. per minute. A preferred rate of cooling is about either 2, 4, or 6 degrees C. per minute. Additionally or alternatively, a treatment device can apply a generally constant pressure during cooling to the supercooled temperature range to avoid pressure changes that would cause inadvertent nucleation. In a further embodiment, the targeted tissue can be cooled while the patient is held still (e.g., without movement of the treatment site) to avoid mechanically disturbing the supercooled tissue and unintentionally causing crystallization. The temperature of the non-targeted surface tissue can be warmed to a non-freezing temperature and/or a non-supercooled temperature prior to perturbation and subsequent freezing. In one embodiment, the warming cycle of the temperature profile can occur quickly such that the underlying and/or targeted tissue remains in the supercooled state throughout the warming cycle.

At least some aspects of the technology are directed to systems and methods of treating a patient by cooling a surface of the patient's skin to a temperature sufficiently low to cause supercooling of targeted tissue below the skin surface. The surface of the skin can then be heated to a non-supercooled temperature while the targeted tissue remains in a supercooled state. After heating the non-targeted tissue, the supercooled targeted tissue can be controllably frozen. In some embodiments, nucleation can be controlled to cause partial freezing. In some procedures, the applicator 104 of FIGS. 1B and 2 can be kept generally stationary relative to the treatment site during cooling to avoid pressure changes that would cause nucleation. After heating non-targeted tissue, the applicator 104 can cause nucleation in the supercooled targeted tissue by, for example, varying applied pressures, delivering energy (e.g., ultrasound energy, RF energy, ultrasound energy, microwave energy, etc.), applying fields (e.g., AC electric fields, DC electric fields, etc.), or providing other perturbations (e.g., vibrations, pulses, etc.), as well as combinations thereof. Because the non-targeted tissue has been warmed to a non-supercooled state, it does not experience a freeze event. In some embodiments, the applicator 104 can include one or more movable plates (e.g., plates movable to vary applied pressures), rotatable eccentric masses, ultrasound transducers, electrical current generators, or other elements capable of providing nucleating perturbations. Vacuum applicators can increase and decrease vacuum levels to massage tissue, vary applied pressures, etc.

Once catalyzed, the partial or total freeze event can be detected, and a cooling device associated with the treatment system 100 can be controlled to continue cooling the patient's skin so as to maintain a frozen state of targeted tissue for a desired period of time. The skin can be periodically or continuously cooled to keep a sufficient volume of the tissue in a frozen state. In some embodiments, the targeted tissue can be kept frozen for longer or shorter than about, for example, 1 second, 5 seconds, 10 seconds, 20 seconds, 30 seconds, 1 minute, several minutes, or other time period selected to reduce or limit frostbite or necrosis. Further, the temperature of the upper tissue of the skin can be detected, and the treatment system 100 can be controlled to apply heat to the surface of the patient's skin for a preselected period of time to prevent freezing of non-targeted tissue. The preselected period of time can be longer or shorter than about 1, 2, 3, 4, or 5 seconds. Accordingly, non-targeted tissue can be protected without using a chemical cryoprotectant that may cause unwanted side effects. Alternatively, a cryoprotectant can also be used if an additional margin of safety for some tissue, such as the epidermis, is desired.

As described herein for targeting subcutaneous lipid-rich tissue, temperature treatment cycles can be used with the treatment system 100 to transdermally cool (e.g., supercool) and selectively affect the patient's subcutaneous lipid-rich tissue while protecting non-lipid rich cells (e.g., residing in epidermal and/or dermal layers) at a temperature higher than the freezing temperatures of the subdermal tissue. Subcutaneous lipid-rich tissue can be supercooled and then frozen for a variety of therapeutic and cosmetic body-contouring applications, such as reduction of adipose tissue residing in identified portions of the patient's body (e.g., chin, cheeks, arms, pectoral areas, thighs, calves, buttocks, abdomen, “love handles”, back, breast, etc.). For example, use of the temperature treatment cycle with the treatment system 100 to transdermally cool adipose tissue in the breast can be used for breast contouring and size reduction in a manner that facilitates protection of non-target tissue in the breast. Further examples include use of the temperature treatment cycle and treatment system 100 to contour and/or reduce a volumetric size of treatment sites without substantially affecting non-targeted cells (e.g., cells in the epidermal and/or dermal layers). In some embodiments, the disclosed methods for therapeutic and cosmetic body-contouring applications can be performed with or without the use of chemical cryoprotectants.

In another embodiment, temperature treatment cycles can be used with the treatment system 100 to cool the skin to selectively affect (e.g., injure, damage, and/or kill) secreting exocrine glandular cells or hair follicles. For example, secreting glandular cells residing in axilla apocrine sweat glands can be targeted by the treatment system 100 for the treatment of hyperhidrosis. In another example, lipid-producing cells residing in or at least proximate to sebaceous glands (e.g., glandular epithelial cells) present in the dermis of a target region can be targeted by the treatment system 100 for the treatment of acne or other skin condition. For example, the treatment system 100 can be configured to reduce a temperature of a dermal layer of skin to reduce the temperature of lipid-producing cells residing in or at least proximate to sebaceous glands such that the targeted lipid-producing cells excrete a lower amount of sebum and/or there are fewer lipid-producing cells resulting in less sebum production within the targeted sebaceous glands. In another embodiment, the sebaceous glands are destroyed. The treatment system 100 can be configured, for example, to reduce a subject's acne by cooling (e.g., supercooling) acne-prone regions of the body (e.g., the face, back and chest).

### 4. Healing of Freeze Wounds

Healing of freeze injuries is different from healing of other types of injuries. For example, significant freezing can destroy cells; however, it does not completely or immediately destroy the surrounding connective tissue matrix. During the healing process, the cell population lost by freeze injury can be replaced by fibroblasts that migrate into the wound site. The degraded matrix and cellular debris at the wound site is removed and replaced gradually, thus enhancing the structural integrity of the wound base, as can be evidenced by a recovering wound site's pattern of birefringence. For example, following freeze injuries, the connective tissue matrix reveals dermal-like patterns of birefringence identical to that which is seen in normal, undamaged skin adjacent to the wound. This is consistent with other findings that the extracellular matrix is relatively resistant to freeze damage. Without being bound by theory, it is believed that wound contraction (e.g., a decrease in the size of the wound) does not occur following a freezing injury because of the relatively intact connective tissue matrix at the wound site.

Freezing will destroy cells (e.g., epidermal keratinocytes), which are then removed by phagocytosis and replaced gradually, but freezing relatively spares the connective tissue matrix which can simulate the effects of a full-thickness skin graft to an open wound. Freezing of the skin can also promote separation between dermal and epidermal layers during the post-thaw period. For example, immediately following a freeze event, epidermal necrosis, pyknotic dermal fibroblasts and polymorphonuclear leukocytes are evident at the wound site. As such, inflammation, granulation tissue formation, and epithelialization are natural processes that occur during freeze wound healing.

During the repair process following freeze injuries, the turnover of connective tissue matrix can be diminished or delayed. The residual matrix does, however, retain much of its structural integrity. This is observed by the absence of degradative denaturation as demonstrated by its patterns of birefringence. There is little collagenous phase change leading to fibrillar shrinkage at this stage.

Wound repair resulting from a freeze event or injury follows a generalized pattern of healing phases that can be categorized depending upon the tissue depth of resulting injury. In one example in which freeze injury is limited to the epithelium, the epithelium restores or regenerates itself to a structure similar to the pre-injury state. In contrast, injuries inflicted on deeper structures or skin layers (e.g., the dermis) typically result in more extensive tissue repair process. For example, in acute partial thickness (e.g., superficial) freeze wounds, epithelialization and/or regeneration occurs by a different mechanism than full-thickness (e.g., deeper) wounds because adnexal structures are retained. These adnexal structures can serve as a reservoir of epithelial cells that migrate across the wound to repopulate the epidermis. Without being bound by theory, and in a particular example of wound healing following a freeze event, a wound healing process may progress through three or more healing phases. The healing phases may include, without limitation, (1) an inflammatory phase, (2) a proliferative phase, and (3) a remodeling phase.

An onset of an inflammatory process can result in localized edema. Although the scars from the freeze-produced wounds can have larger surface areas due to the lack of wound contraction as described above, freeze wounds will, however, often remain more localized due to the absence of thermal radiation dynamics. For example, tissue examined 4 days after freeze injury often reveals edema and inflammatory cells at the interface between dead and surviving dermal tissue, and viable myofibroblasts can be visible within the central wound area associated with the residual connective tissue matrix. Approximately 10 days following a freeze injury or event, the wound site typically reveal islands of granulation tissue intermingled with residual connective tissue matrix within the area of healing. Few viable cells are evident within this residual matrix, but the islands of granulation tissue can contain densely packed myofibroblasts. During an inflammatory phase of healing, platelets are among the first cells to appear at the wound site. Platelets release platelet derived growth factor (PDGF), which upregulates soluble fibrinogen production. Fibrinogen is converted to insoluble strands of fibrin which form a matrix for the influx of monocytes and fibroblasts.

During a proliferative phase of healing, cellular activity promotes epithelialization and fibroplasia. Fibronectin, produced initially from plasma, promotes epidermal migration by providing its own lattice. In freeze wounds, basal keratinocytes secrete collagenase-1 when in contact with fibrillar collagen. Collagenase-1 disrupts attachment to fibrillar collagen which allows for continued migration of keratinocytes into the wound site. It is during the proliferative phase that a healing process following freezing injury can result in a thicker epidermal layer with increased cellular activity.

Extracellular matrix remodeling, cell maturation and cell apoptosis create the remodeling phase of wound repair, which processes can also overlap with tissue formation. Tissue remodeling describes transient to permanent changes in the tissue architecture that involve breaching of histological barriers, such as basement membranes, basal lamina, and extracellular matrix. Typically, the remodeling phase addresses the potential outcome of freeze wound repair as this phase creates structural integrity and textural quality enhancement, which can define the clinical outcome.

Healing of freeze wounds also includes the deposition of matrix materials. Dermal macromolecules, such as fibronectin, hyaluronic acid, proteoglycans and collagen, are deposited and serve as a scaffolding for subsequent cellular migration and tissue support. Deposition and remodeling of the extracellular matrix proteins are dynamic processes and differences in the quantity of matrix proteins are evident between the center and the periphery of the wound. Since the collagen matrix is retained in freeze wounds at or near their pre-injury structural integrity levels, its tensile strength, which is a functional assessment of collagen, can be enhanced. Therefore, the collagen matrix can provide (in conjunction with the increase in epithelial layer thickness) a transient barrier to the pre-injury surface nodularity of cellulite.

### D. Treatment Systems and Methods of Treatment

FIG. 2 is a partially schematic isometric view of the non-invasively treatment system 100 for performing cryotherapy procedures disclosed herein. The term “treatment system”, as used generally herein, refers to cosmetic or medical treatment systems. The treatment system 100 can be configured to alter a human subject's subcutaneous adipose tissue, reduce skin surface irregularities, and/or improve skin characteristics by cooling targeted cells. The treatment system 100 can include a treatment unit or tower 102 (“treatment tower 102”) connected to the applicator 104 by supply and return fluid lines 108a-b and power-lines 109a-b. The applicator 104 can have one or more cooling devices powered by electrical energy delivered via the power-lines 109a-b. A control line can provide communication between electrical components of the applicator 104 and a controller 114. Components of the applicator 104 can be cooled using coolant that flows between the applicator 104 and the treatment tower 102 via the supply and return fluid lines 108a-b. In one example, the applicator 104 has a cooling device (e.g., cooling/heating device 103 of FIG. 1B) with one or more thermoelectric cooling elements and fluid channels through which the coolant flows to cool the thermoelectric cooling elements. The thermoelectric cooling elements can include heat-exchanging plates, Peltier devices, or the like. In other embodiments, the applicator 104 can be a non-thermoelectric device that is heated/cooled using only coolant.

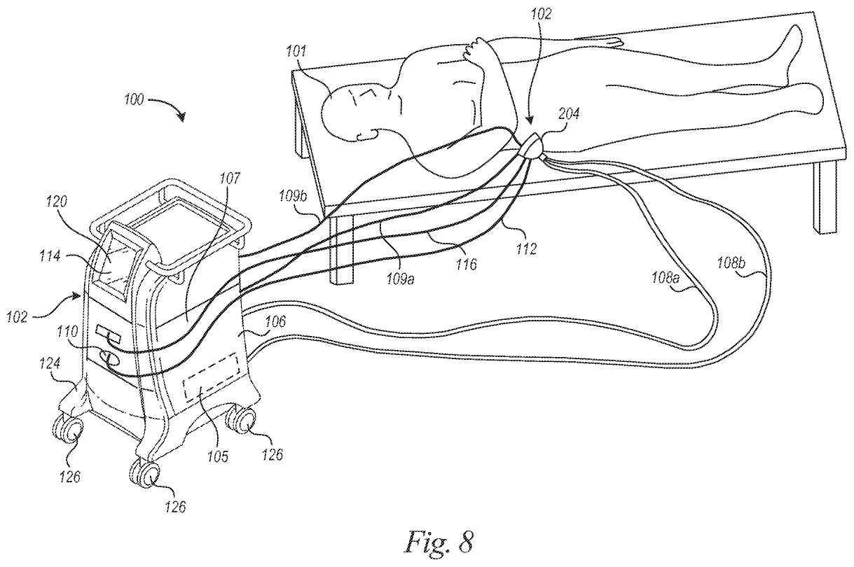

The treatment tower 102 can include a chiller unit or module 106 (“chiller unit 106”) capable of removing heat from the coolant. The chiller unit 106 can include one or more refrigeration units, thermoelectric chillers, or any other cooling devices and, in one embodiment, includes a fluid chamber configured to house the coolant delivered to the applicator 104 via the fluid lines 108a-b. In some procedures, the chiller unit 106 can circulate warm coolant to the applicator 104 during periods of warming. In certain procedures, the chiller unit 106 can alternatingly provide heated and chilled coolant for warming and cooling periods. The circulating coolant can include water, glycol, synthetic heat transfer fluid, oil, a refrigerant, or any other suitable heat conducting fluid. Alternatively, a municipal water supply (e.g., tap water) can be used in place of or in conjunction with the treatment tower 102. The fluid lines 108a-b can be hoses or other conduits constructed from polyethylene, polyvinyl chloride, polyurethane, and/or other materials that can accommodate the particular coolant. One skilled in the art will recognize that there are a number of other cooling technologies that could be used such that the treatment unit, chiller unit, and/or applicator(s) need not be limited to those described herein. Additional features, components, and operation of the treatment tower 102 are discussed in connection with FIG. 8.

FIG. 2 shows the applicator 104 positioned to treat tissue along the leg of the subject 101. Feedback data from sensors of the applicator 104 can be collected in real-time because real-time processing of such feedback data can help correctly and efficaciously administer treatment. In one example, real-time data processing is used to detect freeze events and to control the applicator 104 to continue cooling the patient's skin after the freeze event is detected. Tissue can be monitored to keep the tissue in the frozen state (e.g., a partial or total frozen state) for a period of time. The period of time can be selected by the treatment tower 102 or an operator and can be longer than about, for example, 10 seconds, 30 seconds, 1 minute, or a few minutes. Other periods of time can be selected if needed or desired. The applicator 104 can include sensors configured to measure tissue impedance, pressure applied to the subject 101, optical characteristics of tissue, and/or tissue temperatures. As described herein, sensors can be used to monitor tissue and, in some embodiments, to detect freeze events. The number and types of sensors can be selected based on the treatment to be performed.

Multiple applicators may be concurrently or sequentially used with the treatment system 100 and applied during a treatment session, and such applicators can include, without limitation, vacuum applicators, belt applicators, and so forth. Each applicator may be designed to treat identified portions of the patient's body, such as chin, cheeks, arms, pectoral areas, thighs, calves, buttocks, abdomen, “love handles”, back, and so forth. For example, a vacuum applicator may be applied at the back region, and the belt applicator may be applied around the thigh region, either with or without massage or vibration. Exemplary applicators and their configurations usable or adaptable for use with the treatment system 100 are described in, e.g., U.S. Pat. No. 8,834,547 and commonly assigned U.S. Pat. No. 7,854,754 and U.S. Patent Publication Nos. 2008/0077201, 2008/0077211, and 2008/0287839, which are incorporated by reference in their entireties.

In further embodiments, the system 100 may also include a patient protection device (not shown) incorporated into or configured for use with the applicator 104 that prevents the applicator from directly contacting a patient's skin and thereby reduces the likelihood of cross-contamination between patients and minimizes cleaning requirements for the applicator. The patient protection device may also include or incorporate various storage, computing, and communications devices, such as a radio frequency identification (RFID) component, to monitor and/or meter use. Exemplary patient protection devices are described in commonly assigned U.S. Patent Publication No. 2008/0077201.

In operation, and upon receiving input to start a treatment protocol, the controller 114 can cycle through each segment of a prescribed treatment plan. In so doing, power supply 110 and chiller unit 106 can provide power and coolant to one or more functional components of the applicator 104, such as thermoelectric coolers (e.g., TEC “zones”), to begin a cooling cycle and, in some embodiments, activate features or modes such as vibration, massage, vacuum, etc. The controller 114 can monitor treatment by receiving temperature readings from temperature sensors. The temperature sensors can be part of the applicator 104 or proximate to the applicator 104, the patient's skin, a patient protection device, etc. It will be appreciated that while a target region of the body has been cooled or heated to the target temperature, in actuality that region of the body may be close but not equal to the target temperature, e.g., because of the body's natural heating and cooling variations. Thus, although the system 100 may attempt to heat or cool tissue to the target temperature or to provide a target heat flux, a sensor may measure a sufficiently close temperature or heat flux. If the target temperature or flux has not been reached, power can be increased or decreased to change heat flux to maintain the target temperature or “set-point” selectively to affect targeted tissue. The system 100 can thus monitor the treatment site while accurately cooling/heating to perform the methods disclosed herein.

The applicator 104 can damage, injure, disrupt or otherwise reduce subcutaneous lipid-rich cells generally without collateral damage to non-lipid-rich cells in the treatment region. In other embodiments, the applicator 104 damages, injures, disrupts, or otherwise reduces cells in the epidermal and/or dermal layers to create freeze events (e.g., thermal injuries and/or trauma for achieving desired effects). A cryoprotectant can be administered topically to the skin of the subject 101 at the treatment site and/or used with the applicator 104 to, among other advantages, assist in preventing or, in other embodiments, controlling freezing of targeted cells. Supercooling or other techniques can be performed without the use of topically applied cryoprotectants.

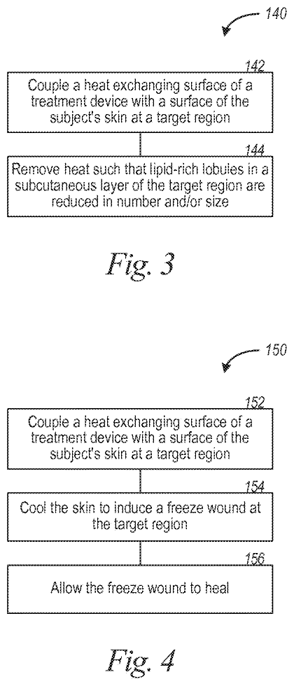

FIGS. 3 to 7 are flow diagrams illustrating methods for treating treatment sites in accordance with embodiments of the technology. Although specific example methods are described herein, one skilled in the art is capable of identifying other methods that the system could perform. Moreover, the methods described herein can be altered in various ways. Even though the methods are described with reference to the treatment system 100 of FIG. 2, the methods may also be applied in other treatment systems with additional or different hardware and/or software components.

FIG. 3 is a flow diagram illustrating a method 140 for reducing irregularities in a surface of a subject's skin resulting from an uneven distribution of adipose tissue in the subcutaneous layer in accordance with embodiments of the technology. As shown in FIG. 3, an early stage of the method 140 can include coupling a heat-exchanging surface of a treatment device with the surface of the subject's skin at a target region (block 142). FIG. 1B shows the heat-exchanging surface 19 in the form of an exposed surface of a heat-exchanging plate thermally coupled to the subject's skin. In another embodiment, the heat-exchanging surface can be the surface of an interface layer or a dielectric layer. Coupling of heat-exchanging surfaces to the skin can be facilitated by using restraining means, such as a belt or strap. In other embodiments, a vacuum or suction force can be used to positively couple the treatment device to the patient's skin. In some methods, a conductive substance can couple the heat-exchanging surface 19 to the patient's skin and can be a cryoprotectant. Cryoprotectants and methods of using cryoprotectants are described in commonly assigned U.S. Patent Publication No. 2007/0255362.

At block 144, the method 140 includes removing heat such that lipid-rich cells in the subcutaneous layer are reduced in number and/or size to an extent while non-lipid-rich cells and lipid-rich regions adjacent to the fibrous septae are not reduced in number or size to the same extent. For example, cooling the subcutaneous layer in the target region can include cooling the lipid-rich tissue to a temperature below, for example, about 10° C., 0° C., −5° C., −10° C., −15° C., −20° C., −25° C., −30° C., −35° C., or −40° C. to disrupt lipid-rich lobules and the adipose cells. The duration of cooling may vary depending on the location of the target region and the degree of cooling required to reduce the number and/or size of the lipid-rich cells, as well as other parameters.

FIG. 4 is a flow diagram illustrating a method 150 for reducing the appearance of cellulite in a target area of a subject in accordance with embodiments of the technology. As shown in FIG. 4, the method 150 can include coupling a heat-exchanging surface of a treatment device with the surface of the subject's skin at a target region (block 152). In one embodiment, the heat-exchanging surface can be a surface of a heat-exchanging plate. In another embodiment, the heat-exchanging surface can be the surface of an interface layer or a dielectric layer. At block 154, the method 150 includes cooling the subject's skin to induce a freeze wound at the target region and allowing the freeze wound to heal (block 156). As discussed above, freezing and thawing events can induce injury to the skin tissue. Such injury can promote natural body responses (e.g., healing) that can have positive effects on skin appearance. For example, and in one embodiment, the method can promote thickening of the epidermal layer to reduce the appearance of cellulite.

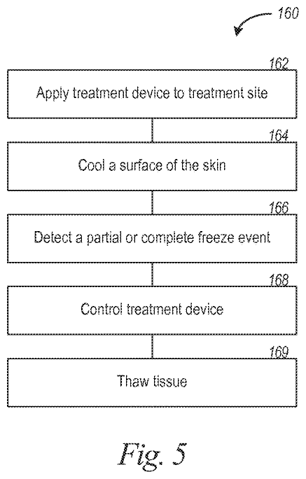

FIG. 5 is a flow diagram illustrating a method 160 for improving the appearance of skin by producing one or more freeze events in accordance with embodiments of the technology. Generally, a treatment device can be applied to a subject and can cool a surface of the subject's skin to produce and detect a freeze event. After detecting the freeze event (or events), operation of the treatment device can be controlled to keep at least a portion of the subject's tissue frozen for a sufficient length of time to improve skin appearance. Details of method 160 are discussed below.

At block 162, the treatment device is applied to a subject by placing its heat-exchanging surface in thermal contact with the subject's skin. A substance can be applied to the subject's skin before applying the treatment device and can (a) provide thermal coupling between the subject's skin and the treatment device to improve heat transfer therebetween, (b) selectively protect non-target tissues from freeze damage (e.g., damage due to crystallization), and/or (c) initiate and/or control freeze events. The substance may be a fluid, a gel, or a paste and may be hygroscopic, thermally conductive, and biocompatible. In some embodiments, the substance can be a cryoprotectant that reduces or inhibits cell destruction. As used herein, “cryoprotectant,” “cryoprotectant agent,” and “composition” mean substances (e.g., compositions, formulations, compounds, etc.) that assist in preventing freezing of tissue compared to an absence of the substances(s). In one embodiment, the cryoprotectant allows, for example, the treatment device to be pre-cooled prior to being applied to the subject for more efficient treatment. Further, the cryoprotectant can also enable the treatment device to be maintained at a desired low temperature while preventing ice formation on the cooled surface of the treatment device, and thus reduces the delay in reapplying the treatment device to the subject. Yet another aspect of the technology is the cryoprotectant may prevent the treatment device from freezing to the subject's skin. Certain cryoprotectant can allow microscopic crystals to form in the tissue but can limit crystal growth that would cause cell destruction and, in some embodiments, allows for enhanced uptake or absorption and/or retention in target tissue prior to and during cooling.

Some embodiments according to the present technology may use a cryoprotectant with a freezing point depressant that can assist in preventing freeze damage that would destroy cells. Suitable cryoprotectants and processes for implementing cryoprotectants are described in commonly-assigned U.S. Patent Publication No. 2007/0255362. The cryoprotectant may additionally include a thickening agent, a pH buffer, a humectant, a surfactant, and/or other additives and adjuvants as described herein. Freezing point depressants may include, for example, propylene glycol (PG), polyethylene glycol (PEG), dimethyl sulfoxide (DMSO), or other suitable alcohol compounds. In a particular embodiment, a cryoprotectant may include about 30% propylene glycol, about 30% glycerin (a humectant), and about 40% ethanol. In another embodiment, the cryoprotectant may include about 40% propylene glycol, about 0.8% hydroxyethyl cellulose (a thickening agent), and about 59.2% water. In a further embodiment, a cryoprotectant may include about 50% polypropylene glycol, about 40% glycerin, and about 10% ethanol. The freezing point depressant may also include ethanol, propanol, iso-propanol, butanol, and/or other suitable alcohol compounds. Certain freezing point depressants (e.g., PG, PPG, PEG, etc.) may also be used to improve spreadability of the cryoprotectant and to provide lubrication. The freezing point depressant may lower the freezing point of body liquids/lipids to about 0° C. to −50° C., about 0° ° C. to −50° C., or about 0° ° C. to −30° C. In other embodiments, the freezing point of the liquid/lipids can be lowered to about −10° C. to about −40° C., about −10° C. to about −30° C., or about −10° C. to about −20° C. In certain embodiments, the freezing point of the liquid/lipids can be lowered to a temperature below about 0° C., below about −5° C., below about −10° C., below about −12° C., below about −15° C., below about −20° C., below about −30° C., or below about −35° C. For example, the freezing point depressant may lower the freezing point of body fluid/lipids to a temperature of between about −1° C. and −40° C., between about −5° C. and −40° C., or between about −10 and −40° C.

Cryoprotectant can be delivered to the surface of the patient's skin for a period of time which is short enough to not significantly inhibit the initiation of the freeze event in dermal tissue but which is long enough to provide substantial protection to non-targeted tissue (e.g., subcutaneous adipose tissue). The rate of cryoprotectant delivery can be selected based on the characteristics of the cryoprotectant and the desired amount of tissue protection. In one specific treatment process, an interface member is placed directly over the target area, and the treatment device with a disposable sleeve or liner is placed in contact with the interface member. The interface member can be a cotton pad, a gauze pad, a pouch, or a container with a reservoir containing a volume of cryoprotectant or other flowable conductive substance. The interface member can include, for example, a non-woven cotton fabric pad saturated with cryoprotectant that is delivered at a desired delivery rate. Suitable pads include Webril™ pads manufactured by Covidien of Mansfield, Massachusetts. Further details regarding interface members and associated systems and methods of use are described in commonly-assigned U.S. Patent Publication No. 2010/0280582.

At block 164, the treatment device can rapidly cool the surface of the patient's skin to a sufficiently low temperature to cause a freeze event in targeted tissue. The rapid cooling can create a thermal gradient with the coldest temperatures near the applicator (e.g., the upper layers of skin). The resulting thermal gradient causes the temperature of the upper layer(s) of the skin to be lower than that of the targeted deeper cells. This allows the skin to be frozen for a short enough duration to not establish a temperature equilibrium across the skin and adjacent subcutaneous tissue. A cryoprotectant and/or warming cycle can be used to inhibit freezing the uppermost non-targeted layer or layers of skin.

A freeze event can include at least some crystallization (e.g., formation of microscopic ice crystals) in intercellular material (e.g., fluid, cell components, etc.) and/or extracellular fluid. By avoiding extensive ice crystal formation that would cause frostbite or necrosis, partial freeze events can occur without undesired tissue damage. In some embodiments, the surface of the patient's skin can be cooled to a temperature no lower than about −40° C., −30° C., −20° C., −10° C., or −5° C. to produce a partial or total freeze event without causing irreversible skin damage. In one example, the treatment system 100 of FIG. 2 can cool the surface of the skin to from about −40° C. to about 0° C., from about −30° C. to about 0° C., from about −20° C. to about 0° C., or from about −15° C. to about 0° C. or below about −10° C., −20° C., −20° C., −30° C., or −40° C. It will be appreciated that the surface of skin can be cooled to other temperatures based on the mechanism of action.

The cooling period can be sufficiently short to minimize, limit, or substantially eliminate necrosis, or other unwanted thermal damage, due to the freeze event. In one procedure, the applicator (e.g., applicator 104 of FIGS. 1B and 2) can produce a freeze event that begins within a predetermined period of time after the applicator begins cooling the patient's skin or after the freeze event begins. The predetermined period of time can be equal to or shorter than about 30, 60, 90, 120, or 150 seconds and, in some embodiments, the predetermined period of time can be from between about 10 seconds to about 150 seconds, between about 30 seconds to about 150 seconds, or between about 60 seconds to about 150 seconds. In some embodiments, the predetermined period of time can be shorter than about either 1, 2, 3, 4, 5, 6, 7, 8, 9, or 10 minutes. A controller (e.g., controller 114 of FIG. 2) can select the predetermined period of time based on the treatment temperatures, treatment sites, and/or cryotherapy to be performed. Alternatively, an operator can select the period of time for cooling and can enter it into the controller 114.

To help initiate the freeze event (e.g., the partial or total freeze event), energy, pressure, and/or a substance can be used to aid in the formation of nucleation sites for crystallization. Energy for promoting nucleation can include, without limitation, acoustic energy (e.g., ultrasound energy), mechanical energy (e.g., vibratory motion, massaging, and/or pulsatile forces), or other suitable energy. The applicators disclosed herein can include, without limitation, one or more elements (e.g., elements 171 in FIG. 1B) in the form of actuators (e.g., motors with eccentric weights), vibratory motors, hydraulic motors, electric motors, AC electrodes, pneumatic motors, solenoids, piezoelectric shakers, and so on for providing energy, pressure, etc. Pressure for promoting nucleation can be applied uniformly or non-uniformly across the treatment site. Substances that promote nucleation can be applied topically before and/or during skin cooling.

At block 166 of FIG. 5, the treatment device can detect the freeze event using one or more electrical components. FIG. 1B shows the applicator 104 with an electronic component in the form of a sensor 167 that can identify positive (increase) or negative (decrease) temperature changes. During cooling, targeted tissue can reach a temperature below the freezing point of its biological tissue and fluids (e.g., approximately −1.8° C.). As tissue, fluids, and lipids freeze, crystals can form and energy associated with the latent heat of crystallization is released. A relatively small positive change in tissue temperature can indicate a partial freeze event whereas a relatively large positive change in tissue temperature can indicate a complete freeze event. The sensor 167 (FIG. 1B) can detect the positive change in tissue temperature, and the treatment system can identify it as a freeze event. The treatment system can be programmed so that small temperature variations do not cause false alarms with respect to false treatment events. Additionally or alternatively, the treatment systems disclosed herein may detect changes in the temperature of its components or changes in power supplied to treatment devices, or other components, to identify freeze events.

Referring now to FIG. 2, the system 100 can monitor the location and/or movement of the applicator 104 and may prevent false or inaccurate determinations of treatment events based on such monitoring. The applicator 104 may move during treatment which may cause the applicator 104 to contact a warmer area of skin, to no longer contact the skin, and so on. This may cause the system 100 to register a difference in temperature that is inconsistent with a normal treatment. The controller 114 may be programmed to differentiate between these types of temperature increases and a temperature increase associated with freezing. U.S. Pat. No. 8,285,390 discloses techniques for monitoring and detecting freeze events and applicator movement and is incorporated by reference in its entirety. Additionally, the treatment system 100 can provide an indication or alarm to alert the operator to the source of this temperature increase. In the case of a temperature increase not associated with a treatment event, the system 100 may also suppress false indications, while in the case of a temperature increase associated with freezing, the system 100 take any number of actions based on that detection as described elsewhere herein.

The system 100 can use optical techniques to detect events at block 166 of FIG. 5. For example, the sensor 167 of FIG. 1B can be an optical sensor capable of detecting changes in the optical characteristics of tissue caused by freezing. The optical sensor can include one or more energy emitters (e.g., light sources, light emitting diodes, etc.), detector elements (e.g., light detectors), or other components for non-invasively monitoring optical characteristics of tissue. In place of or in conjunction with monitoring using optical techniques, tissue can be monitored using electrical and/or mechanical techniques. In embodiments for measuring electrical impedance of tissue, the sensor 167 (FIG. 1B) can include two electrodes that can be placed in electrical communication with the skin for monitoring electrical energy traveling between the electrodes via the tissue. In embodiments for measuring mechanical properties of tissue, the sensor 167 can comprise one or more mechanical sensors which can include, without limitation, force sensors, pressure sensors, and so on.

At block 168 of FIG. 5, the treatment device can be controlled to maintain the freeze event by continuously or periodically cooling the patient's tissue to keep a target volume of skin frozen for a period of time, which can be long enough to improve skin appearance. In short treatments, the period of time can be equal to or shorter than about 5, 10, 15, 20, or 25 seconds. In longer treatments, the period of time can be equal to or longer than about 25 seconds, 30 seconds, 45 seconds or 1, 2, 3, 4, 5, or 10 minutes. In some procedures, the applicator 104 of FIGS. 1B and 2 can be controlled so that the skin is partially or completely frozen for no longer than, for example, 5 minutes, 10 minutes, 20 minutes, 30 minutes, 45 minutes, or 1 hour. In some examples, the skin is frozen for about 1 minute to about 5 minutes, about 5 minutes to about 10 minutes, about 10 minutes to about 20 minutes, about 20 minutes to about 30 minutes, or about 30 minutes to about 1 hour. The length of time the skin is kept frozen can be selected based on the severity of the freeze injury.

At block 168, the treatment system can also control the applicator so that the partial or total freeze event causes apoptotic damage to targeted tissue but does not cause such damage to non-targeted tissue. In one example, the applicator produces a partial freeze event short enough to prevent establishing equilibrium temperature gradients in the patient's skin during, for example, the freeze event. This allows freezing of shallow targeted tissue without substantially affecting deeper non-targeted tissue. Moreover, cells in the dermal tissue can be affected to a greater extent than the cells in the subcutaneous layer. In some procedures, the subcutaneous layer can be kept at a sufficiently high temperature (e.g., at or above 0° C.) while the shallower dermal tissue experiences the partial or total freeze event. The system can also control operation of the applicator to thermally injure tissue to cause fibrosis, which increases the amount of connective tissue in a desired tissue layer (e.g., epidermis and/or dermis) to increase the firmness and appearance of the skin. In other treatments, the system controls the applicator to supercool and then freeze (e.g., partially or totally freeze) at least a portion of subcutaneous tissue, such as the fibrous septae.

At block 169, the patient's partially or completely frozen tissue can be thawed by heating it in order to minimize, reduce, or limit tissue damage. The applicator can thaw the patient's skin, or other frozen tissue, after the freeze event occurs and after a period of time has transpired. The period of time can be equal to or shorter than about 5, 10, 15, 20, or 25 seconds or about 1, 2, 3, 4, 5, 6, 7, 8, 9, or 10 minutes. In one example, the uppermost skin layer(s) can be periodically heated to a temperature above the skin's freezing point to provide freeze protection thereto. The applicator can include one or more thermal elements (e.g., resistive heaters, electromagnetic energy emitters, Peltier devices, etc.) for heating frozen tissue. For example, the element 103 of FIG. 1B can be a Peltier device capable of generating heat for thawing tissue. Alternatively, the applicator 104 can include one or more resistive heaters or Peltier devices 171 for thawing tissue. In some embodiments, the applicator 104 of FIGS. 1B and 2 can have separate and independently controlled cooling elements and heating elements that can cooperate to provide precise temperature control for freezing and thawing/warming cycles. In some embodiments, the applicator may stop cooling tissue to allow cooled tissue to naturally warm and thaw.

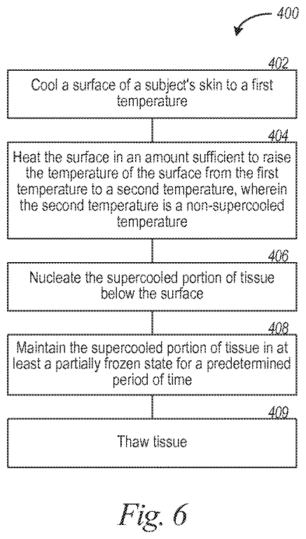

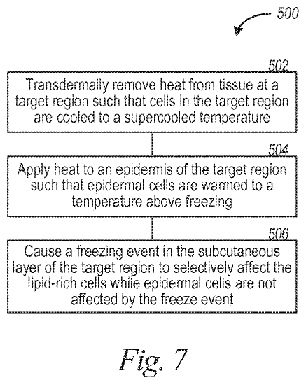

FIGS. 6 and 7 are flow diagrams illustrating methods for supercooling tissue in accordance with embodiments of the technology. Generally, the methods can include treating a human subject's body to cool a surface of the subject's skin to a temperature no lower than −40° C. to avoid unwanted skin damage and so that the temperature of at least a portion of tissue below the surface is in a supercooled state. The surface of the skin can be heated to bring shallow non-targeted tissue out of the supercooled state while deeper targeted tissue remains in the supercooled state. The supercooled targeted tissue can be nucleated due to a perturbation that causes at least partial freezing that destroys or damages targeted cells due to crystallization of intracellular and/or extracellular fluids. The perturbation can be vibrations, ultrasound pulses, and/or changes in pressure suitable for inducing a partial or complete freeze event to disrupt or destroy targeted lipid-rich cells. The treatment system 100 (FIGS. 2 and 8) can utilize applicators disclosed herein to perform such supercooling method.

FIG. 6 is a flow diagram illustrating a method 400 in accordance with an aspect of the present technology. An early stage of the method 400 can include cooling a surface of a human subject's skin to a first temperature (block 402). The first temperature can be, for example, between about −10° C. and −40° C. such that a portion of tissue below the surface is in a supercooled state. In other embodiments, the first temperature can be a temperature between about −15° ° C. and −25° C., a temperature between about −20° C. and about −30° C., or other temperature below a freezing temperature.

In block 404, the surface of the human subject's skin is heated an amount sufficient to raise the skin surface temperature from the first temperature to a second temperature, which can be a non-supercooled temperature, while the targeted tissue remains in the supercooled state. For example, the treatment system can be used to heat the surface (e.g., the epidermis) of the skin to a temperature higher than about 0° C., higher than about 5° C., higher than about 10° C., higher than about 20° C., higher than about 30° C., or higher than about 35° C. There can be a temperature gradient between the targeted tissue and the skin surface such that most of the non-targeted tissue is at a non-supercooled temperature.

In block 406, the supercooled portion of tissue below the skin surface can be nucleated to cause at least some fluid and cells in the supercooled tissue to at least partially freeze. In one embodiment, nucleation of the supercooled tissue is caused by a mechanical perturbation. Cells residing at the surface of the human subject's skin do not freeze, and in certain arrangements, protection of cells at the surface can be accomplished without the use of a chemical cryoprotectant.

In block 408, the supercooled tissue can be maintained in the at least partially or totally frozen state for a predetermined period of time longer than, for example, about 10 seconds, 12 seconds, 15 seconds, or 20 seconds. In various arrangements, the supercooled tissue can be maintained in the at least or totally frozen state for a duration of time sufficient to improve an appearance of skin (e.g., by tightening the skin, increasing skin smoothness, thickening the skin, improving the appearance of cellulite, etc.), treat acne, improve a quality of hair, improve a condition associated with hyperhidrosis, etc. In certain embodiments, the maintaining step can include detecting the temperatures and controlling the cooling and heating to maintain targeted tissue in at least a partially or totally frozen state for the predetermined time (e.g., longer than about 10 seconds, longer than about 12 seconds, longer than about 15 seconds, or longer than about 20 seconds).

In block 409, the patient's partially or completely frozen tissue can be optionally thawed by heating it in order to minimize, reduce, or limit tissue damage. The applicator can thaw the patient's skin, or other frozen tissue, after the freeze event occurs and after a period of time has transpired. In some embodiments, the applicator may stop cooling tissue to allow cooled tissue to naturally warm and thaw.

The method 400 can be performed to keep the freeze event localized in the targeted layer. After supercooling tissue, epidermal tissue can be heated to prevent freeze injuries to the epidermal cells. In other embodiments, the freeze event can be centered at the interface between the dermis and the subcutaneous layer or at any other location. The method 400 can be repeated any number of times at the same location or different locations along the subject.

FIG. 7 illustrates a method 500 for affecting a subcutaneous layer of a human subject's body in accordance with another embodiment of the present technology. The method 500 can include transdermally removing heat from tissue at a target region such that cells in the target region are cooled to a supercooled temperature (block 502). The supercooled temperature can be, for example, below about 0° C. or within a range from about 0° C. to about −20° C., from about −10° C. to about −30° C., from about −20° C. to about −40° C., or no lower than about −40° C. Cryoprotectants can be used to cool tissue to very low temperatures, including temperatures lower than −40° C.

In block 504, the method 500 includes applying heat to an epidermis of the target region to warm epidermal cells in the target region to a temperature above freezing while lipid-rich cells in the subcutaneous layer of the target region are at or near the supercooled temperature. For example, the step of applying heat can include warming the epidermal cells to a temperature above about 5° C., about 10° C., about 20° C., about 25° C., or about 32° C.

In bock 506, a freeze event in the subcutaneous layer of the target region can selectively affect the lipid-rich cells while epidermal cells are not affected by the freeze event. The method 500 can include providing at least one of vibration, mechanical pressure, and ultrasound pulses to the target region to cause such freeze event. In various arrangements, the freeze event can cause at least partial crystallization of a plurality of lipid-rich cells in the target region. Beneficially, the epidermal cells are protected such that freeze damage to these cells does not occur. In certain embodiments, freeze damage protection of the epidermal tissue can occur without applying a cryoprotectant to the surface of the skin prior to or during the treatment.

In some embodiments, the dermal layer can be supercooled while the subcutaneous layer can remain above its freezing point to avoid affecting the subcutaneous lipid-rich cells. The freeze event can occur in the dermal layer after non-targeted epidermal tissue has been warmed such that freeze induced changes at the target region can be localized in the dermal layer. Thus, dermal cells can be affected without appreciably affecting epidermal and subcutaneous cells. The method 500 can also be modified to produce freeze events in other layers of tissue. For example, a freeze event can be produced within one or more targeted epidermal layers by supercooling the targeted epidermal layer(s) and then warming the non-targeted epidermal layer(s). The supercooled epidermal layers are then nucleated.

Various aspects of the methods 400 (FIG. 6) and 500 (FIG. 7) can include cosmetic treatment methods for treating the target region of a human subject's body to achieve a cosmetically beneficial alteration of a portion of tissue within the target region. Such cosmetic methods can be administered by a non-medically trained person. The methods disclosed herein can also be used to (a) improve the appearance of skin by tightening the skin, improving skin tone and texture, eliminating or reducing wrinkles, increasing skin smoothness, thickening the skin, (b) improve the appearance of cellulite, and/or (c) treat sebaceous glands, hair follicles, and/or sweat glands.

### E. Treatment Systems and Treatment Devices

FIG. 8 is a partially schematic isometric view of the system 100 with a multi-modality applicator 204 positioned along the subject's waist. The power supply 110 can provide a direct current voltage to the applicator 204 to remove heat from the subject 101. The controller 114 can monitor process parameters via sensors (e.g., sensors of the applicator 204 and/or sensors placed proximate to the applicator 204) via the control line 116 to, among other things, adjust the heat removal rate and/or energy delivery rate based on a custom treatment profile or patient-specific treatment plan, such as those described, for example, in commonly assigned U.S. Pat. No. 8,275,442.

The controller 114 can exchange data with the applicator 204 via an electrical line 112 or, alternatively, via a wireless or an optical communication link. The control line 116 and electrical line 112 are shown without any support structure. Alternatively, control line 116 and electrical line 112 (and other lines including, but not limited to fluid lines 108a-b and power lines 109a-b) may be bundled into or otherwise accompanied by a conduit or the like to protect such lines, enhance ergonomic comfort, minimize unwanted motion (and thus potential inefficient removal of heat from and/or delivery of energy to subject 101), and to provide an aesthetic appearance to the system 100. Examples of such a conduit include a flexible polymeric, fabric, composite sheath, an adjustable arm, etc. Such a conduit (not shown) may be designed (via adjustable joints, etc.) to “set” the conduit in place for the treatment of the subject 101.

The controller 114 can receive data from an input/output device 120, transmit data to a remote output device (e.g., a computer), and/or exchange data with another device. The input/output device 120 can include a display or touch screen (shown), a printer, video monitor, a medium reader, an audio device such as a speaker, any combination thereof, and any other device or devices suitable for providing user feedback. In the embodiment of FIG. 8, the input/output device 120 can be a touch screen that provides both an input and output functionality. The treatment tower 102 can include visual indicator devices or controls (e.g., indicator lights, numerical displays, etc.) and/or audio indicator devices or controls. These features can be part of a control panel that may be separate from the input/output device 120, may be integrated with one or more of the devices, may be partially integrated with one or more of the devices, may be in another location, and so on. In alternative examples, input/output device 120 or parts thereof (described herein) may be contained in, attached to, or integrated with the applicator 204

The controller 114, power supply 110, chiller unit 106 with a reservoir 105, and input/output device 120 are carried by a rack 124 with wheels 126 for portability. In alternative embodiments, the controller 114 can be contained in, attached to, or integrated with the multi-modality applicator 204 and/or a patient protection device. In yet other embodiments, the various components can be fixedly installed at a treatment site. Further details with respect to components and/or operation of applicators, treatment tower, and other components may be found in commonly-assigned U.S. Patent Publication No. 2008/0287839.

The system 100 can include an energy-generating unit 107 for applying energy to the target region, for example, to further interrogate cooled or heated cells via power-lines 109a-b. In one embodiment, the energy-generating unit 107 can be a pulse generator, such as a high voltage or low voltage pulse generator, capable of generating and delivering a high or low voltage current, respectively, through the power lines 109a, 109b to one or more electrodes (e.g., cathode, anode, etc.) in the applicator 204. In other embodiments, the energy-generating unit 107 can include a variable powered RF generator capable of generating and delivering RF energy, such as RF pulses, through the power lines 109a, 109b or to other power lines (not shown). RF energy can be directed to non-targeted tissue to help isolate cooling. For example, RF energy can be delivered to non-targeted tissue, such as epidermal tissue, to inhibit or prevent damage to such non-targeted tissue. In a further embodiment, the energy-generating unit 107 can include a microwave pulse generator, an ultrasound pulse laser generator, or high frequency ultrasound (HIFU) phased signal generator, or other energy generator suitable for applying energy. In additional embodiments, the system 100 can include more than one energy-generating unit 107 such as any one of a combination of the energy modality generating units described herein. Systems having energy-generating units and applicators having one or more electrodes are described in commonly assigned U.S. Patent Publication No. 2012/0022518 and U.S. patent application Ser. No. 13/830,413.

The applicator 204 can include one or more heat-exchanging units. Each heat-exchanging unit can include or be associated with one or more Peltier-type thermoelectric elements, and the applicator 204 can have multiple individually controlled heat-exchanging zones (e.g., between 1 and 50, between 10 and 45; between 15 and 21, etc.) to create a custom spatial cooling profile and/or a time-varying cooling profile. Each custom treatment profile can include one or more segments, and each segment can include a specified duration, a target temperature, and control parameters for features such as vibration, massage, vacuum, and other treatment modes. Applicators having multiple individually controlled heat-exchanging units are described in commonly assigned U.S. Patent Publication Nos. 2008/0077211 and 2011/0238051.

The applicator 204 can be applied with pressure or with a vacuum type force to the subject's skin. Pressing against the skin can be advantageous to achieve efficient treatment. In general, the subject 101 has an internal body temperature of about 37° C., and the blood circulation is one mechanism for maintaining a constant body temperature. As a result, blood flow through the tissue to be treated can be viewed as a heat source that counteracts the cooling of the desired targeted tissue. As such, cooling the tissue of interest requires not only removing the heat from such tissue but also that of the blood circulating through this tissue. Thus, temporarily reducing or eliminating blood flow through the treatment region, by means such as, e.g., applying the applicator with pressure, can improve the efficiency of tissue cooling (e.g., tissue cooling to reduce cellulite, wrinkles, sagging skin, loose skin, etc.) and avoid excessive heat loss. Additionally, a vacuum can pull tissue away from the body which can assist in cooling targeted tissue.

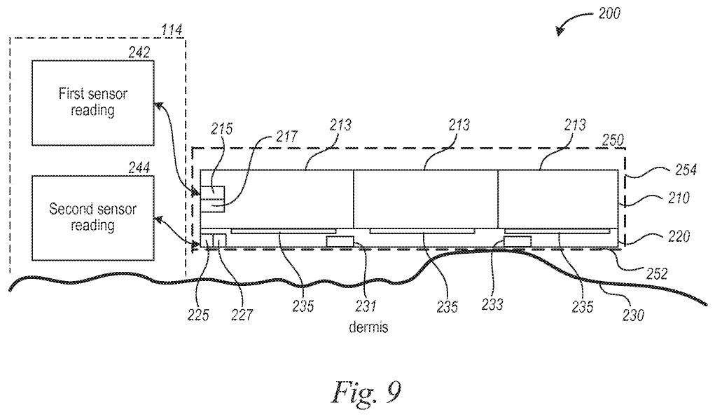

FIG. 9 is a schematic cross-sectional view illustrating a treatment device in the form of an applicator 200 for non-invasively removing heat from target tissue in accordance with an embodiment of the present technology. The applicator 200 can include a cooling device 210 and an interface layer 220. In one embodiment, the cooling device 210 includes one or more thermoelectric elements 213 (e.g., Peltier-type TEC elements) powered by a treatment tower (e.g., treatment tower 102 of FIGS. 2 and 8).

The applicator 200 can contain a communication component 215 that communicates with the controller 114 to provide a first sensor reading 242, and a sensor 217 that measures, e.g., temperature of the cooling device 210, heat flux across a surface of or plane within the cooling device 210, tissue impedance, application force, tissue characteristics (e.g., optical characteristics), etc. The interface layer 220 can be a plate, a film, a covering, a sleeve, a substance reservoir or other suitable element described herein and, in some embodiments, may serve as the patient protection device described herein.

The interface layer 220 can also contain a similar communication component 225 that communicates with the controller 114 to provide a second sensor reading 244 and a sensor 227 that measures, e.g., the skin temperature, temperature of the interface layer 220, heat flux across a surface of or plane within the interface layer 220, contact pressure with the skin 230 of the patient, etc. For example, one or both of the communication components 215, 225 can receive and transmit information from the controller 114, such as temperature and/or heat flux information as determined by one or both of sensors 217, 227. The sensors 217, 227 are configured to measure a parameter of the interface without substantially impeding heat transfer between the cooling device 210 and the patient's skin 230. The applicator 200 can also contain components described in connection with FIGS. 2 and 8.

In certain embodiments, the applicator 200 can include a sleeve or liner 250 (shown schematically in phantom line) for contacting the patient's skin 230, for example, to prevent direct contact between the applicator 200 and the patient's skin 230, and thereby reduce the likelihood of cross-contamination between patients, minimize cleaning requirements for the applicator 200, etc. The sleeve 250 can include a first sleeve portion 252 and a second sleeve portion 254 extending from the first sleeve portion. The first sleeve portion 252 can contact and/or facilitate the contact of the applicator 200 with the patient's skin 230, while the second sleeve portion 254 can be an isolation layer extending from the first sleeve portion 252. The second sleeve portion 254 can be constructed from latex, rubber, nylon, Kevlar®, or other substantially impermeable or semi-permeable material. The second sleeve portion 254 can prevent contact between the patient's skin 230 and the cooling device 210, among other things. Further details regarding a patient protection device may be found in U.S. Patent Publication No. 2008/0077201.

A device (not shown) can assists in maintaining contact between the applicator 200 (such as via an interface layer 220) and the patient's skin 230. The applicator 200 can include a belt or other retention devices (not shown) for holding the applicator 200 against the skin 230. A belt may be rotatably connected to the applicator 200 by a plurality of coupling elements that can be, for example, pins, ball joints, bearings, or other type of rotatable joints. Alternatively, retention devices can be rigidly affixed to the end portions of the interface layer 220. Further details regarding suitable belt devices or retention devices may be found in U.S. Patent Publication No. 2008/0077211.

A vacuum can assist in providing contact between the applicator 200 (such as via the interface layer 220 or sleeve 250) and the patient's skin 230. The applicator 200 can provide mechanical energy to a treatment region using the vacuum. Imparting mechanical vibratory energy to the patient's tissue by repeatedly applying and releasing (or reducing) the vacuum, for instance, creates a massage action during treatment. Further details regarding vacuums and vacuum type devices may be found in U.S. Patent Application Publication No. 2008/0287839.

Optionally, the applicator 200 can include one or more features used with supercooling. For example, the interface layer 220 can include one or more nucleation elements 231, 233 in the form of positive and negative electrodes for heating the skin using alternating current heating. For radiofrequency induced nucleation, the nucleation elements 231, 233 can be RF electrodes. The power supply 110 (FIG. 8) of the treatment tower 102 can include an RF generator for driving the nucleation elements 231, 233. The nucleation elements 231, 233 can also be configured to provide changes in applied pressure to cause nucleation. Any number of different types of nucleation elements can be incorporated into the interface layer 220 or other components of the applicator 200 to provide the ability to controllably nucleate supercooled tissue.

Although the thermoelectric elements 213 can heat tissue, the applicator 200 can also include dedicated heating elements used to, for example, thaw tissue. The interface layer 220 or other components of the applicator 200 can include one or more heaters 235 for generating heat delivered to the surface of the skin 230. The heaters 235 can be resistive heaters, Peltier devices, or other thermoelectric elements capable of generating heat. Optionally, the nucleation elements 231, 233 can also be used to control the temperature of the skin 230. For example, the nucleation elements 231, 233 can include RF electrodes that cooperate to deliver RF energy to heat the skin 230 or deeper tissue.

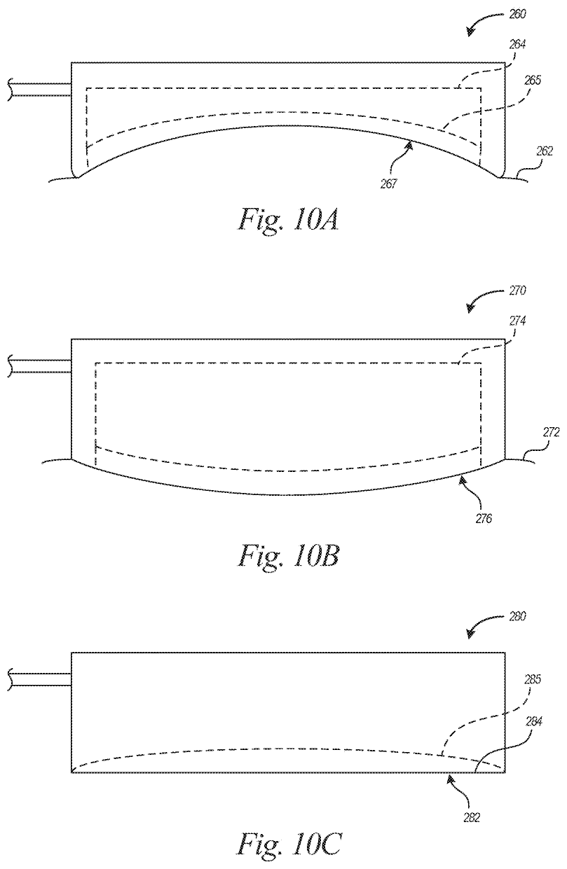

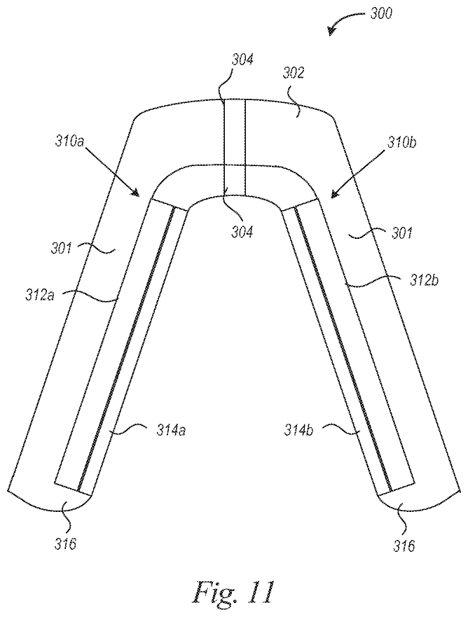

FIGS. 10A to 10C illustrate treatment devices suitable for use with the treatment systems in accordance with embodiments of the technology. FIG. 10A is a schematic, cross-sectional view illustrating an applicator 260 for non-invasively removing heat from target areas of a subject 262. The applicator 260 can include a heat-exchanging unit or cooling device, such as a heat-exchanging plate 264 (shown in phantom line) and an interface layer 265 (shown in phantom line). The interface layer 265 can have a rigid or compliant concave surface 267. When the applicator 260 is held against the subject, the subject's tissue can be pressed against the curved surface 267. One or more vacuum ports can be positioned along the surface 267 to draw the skin 262 against the surface 267. The configuration (e.g., dimensions, curvature, etc.) of the applicator 260 can be selected based on the treatment site.

FIG. 10B is a schematic, cross-sectional view illustrating an applicator 270 that includes a heat-exchanging unit 274 having a rigid or compliant convex surface 276 configured to be applied to concave regions of the subject. Advantageously, the convex surface 276 can spread tissue to reduce the distance between the convex surface 276 and targeted tissue under the convex surface 276.

FIG. 10C is a schematic, cross-sectional view illustrating an applicator 280 including a surface 282 movable between a planar configuration 284 and a non-planar configuration 285 (shown in phantom). The surface 282 is capable of conforming to the treatment site to provide a large contact area. In some embodiments, the surface 282 can be sufficiently compliant to conform to highly contoured regions of a subject's face when the applicator 280 is pressed against facial tissue. In other embodiments, the applicator 280 can include actuators or other devices configured to move the surface 282 to a concave configuration, a convex configuration, or the like. The surface 282 can be reconfigured to treat different treatment sites of the same subject or multiple subjects.

FIG. 11 is a schematic, cross-sectional view of an applicator 300 for non-invasively removing heat from target areas in accordance with another embodiment of the technology. The applicator 300 includes a housing 301 having a vacuum cup 302 with a vacuum port 304 disposed in the vacuum cup 302. The housing 301 is coupled to or otherwise supports a first applicator unit 310a on one side of the cup 302, and a second applicator unit 310b on an opposing side of the cup 302. Each of the first and second applicator units 310a, 310b can include a heat-exchanging unit (e.g., a cooling unit, heating/cooling device, etc.) with a heat-exchanging plate 312 (shown individually as 312a and 312b), and an interface layer 314 (shown individually as 314a and 314b). In one embodiment, the heat-exchanging plate 312 is associated with one or more Peltier-type TEC elements supplied with coolant and power from the treatment tower 102 (FIGS. 2 and 6). As such, the heat-exchanging plates 312a, 312b can be similar to the cooling device 210 described above with reference to FIG. 9.

The interface layers 314a and 314b are adjacent to the heat-exchanging plates 312a and 312b, respectively. Similar to the interface layer 220 illustrated in FIG. 9, the interface layers 314a and 314b can be plates, films, a covering, a sleeve, a reservoir or other suitable element located between the heat-exchanging plates 312a and 312b and the skin (not shown) of a subject. In one embodiment, the interface layers 314a and 314b can serve as patient protection devices and can include communication components (not shown) and sensors (not shown) similar to those described with respect to the interface layer 220 of FIG. 9 for communicating with the controller 114. In other embodiments, the interface layers 314 can be eliminated.

In operation, a rim 316 of the vacuum cup 302 is placed against the skin of a subject and a vacuum is drawn within the cup 302. The vacuum pulls the tissue of the subject into the cup 302 and coapts the target area with the interface layers 314a and 314b of the corresponding first and second applicator units 310a, 310b. One suitable vacuum cup 302 with cooling units is described in U.S. Pat. No. 7,367,341. The vacuum can stretch or otherwise mechanically challenge skin. Applying the applicator 300 with pressure or with a vacuum type force to the subject's skin or pressing against the skin can be advantageous to achieve efficient treatment. The vacuum can be used to damage (e.g., via mechanically massage) and/or stretch connective tissue, thereby lengthen the connective tissue. In general, the subject has an internal body temperature of about 37° C., and the blood circulation is one mechanism for maintaining a constant body temperature. As a result, blood flow through the skin and subcutaneous layer of the region to be treated can be viewed as a heat source that counteracts the cooling of the desired targeted tissue. As such, cooling the tissue of interest requires not only removing the heat from such tissue but also that of the blood circulating through this tissue. Temporarily reducing or eliminating blood flow through the treatment region, by means such as, e.g., applying the applicator with pressure, can improve the efficiency of tissue cooling and avoid excessive heat loss through the dermis and epidermis. Additionally, a vacuum can pull skin away from the body which can assist in cooling targeted tissue.

The units 310a and 310b can be in communication with a controller (e.g., controller 114 of FIGS. 2 and 8), and a supply such that the heat-exchanging plates 312a, 312b can provide cooling or energy to the target region based on a predetermined or real-time determined treatment protocol. For example, the heat-exchanging plates 312a, 312b can first be cooled to cool the adjacent tissue of the target region to a temperature below 37° C. (e.g., to a temperature in the range of between about −40° C. to about 20° C.). The heat-exchanging plates 312a, 312b can be cooled using Peltier devices, cooling channels (e.g., channels through which a chilled fluid flows), cryogenic fluids, or other similar cooling techniques. In one embodiment, the heat-exchanging plates 312a, 312b are cooled to a desired treatment temperature (e.g., −40° C., −30° C., −25° C., −20° C., −18° C., −15° C., −10° C., −5° C., 0° C., or 5° C.). In this example, the lipid-rich cells can be maintained at a sufficiently low temperature to be damaged or destroyed.

### F. Suitable Computing Environments

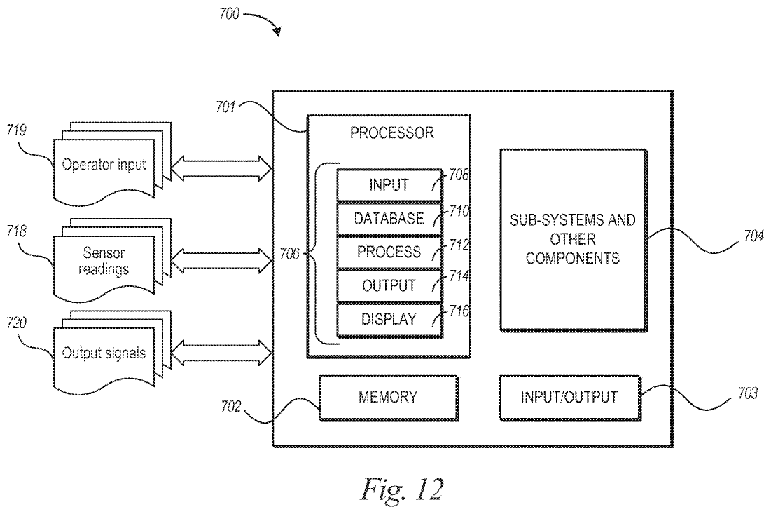

FIG. 12 is a schematic block diagram illustrating subcomponents of a computing device 700 suitable for the system 100 of FIGS. 2 and 8 in accordance with an embodiment of the disclosure. The computing device 700 can include a processor 701, a memory 702 (e.g., SRAM, DRAM, flash, or other memory devices), input/output devices 703, and/or subsystems and other components 704. The computing device 700 can perform any of a wide variety of computing processing, storage, sensing, imaging, and/or other functions. Components of the computing device 700 may be housed in a single unit or distributed over multiple, interconnected units (e.g., though a communications network). The components of the computing device 700 can accordingly include local and/or remote memory storage devices and any of a wide variety of computer-readable media.

As illustrated in FIG. 12, the processor 701 can include a plurality of functional modules 706, such as software modules, for execution by the processor 701. The various implementations of source code (i.e., in a conventional programming language) can be stored on a computer-readable storage medium or can be embodied on a transmission medium in a carrier wave. The modules 706 of the processor can include an input module 708, a database module 710, a process module 712, an output module 714, and, optionally, a display module 716.

In operation, the input module 708 accepts an operator input 719 via the one or more input/output devices described above with respect to FIG. 6, and communicates the accepted information or selections to other components for further processing. The database module 710 organizes records, including patient records, treatment data sets, treatment profiles and operating records and other operator activities, and facilitates storing and retrieving of these records to and from a data storage device (e.g., internal memory 702, an external database, etc.). Any type of database organization can be utilized, including a flat file system, hierarchical database, relational database, distributed database, etc.

In the illustrated example, the process module 712 can generate control variables based on sensor readings 718 from sensors (e.g., sensor 167 of FIG. 1B, the temperature measurement components 217 and 227 of FIG. 9, etc.) and/or other data sources, and the output module 714 can communicate operator input to external computing devices and control variables to the controller 114 (FIGS. 2, 8, and 9). The display module 816 can be configured to convert and transmit processing parameters, sensor readings 818, output signals 720, input data, treatment profiles and prescribed operational parameters through one or more connected display devices, such as a display screen, printer, speaker system, etc. A suitable display module 716 may include a video driver that enables the controller 114 to display the sensor readings 718 or other status of treatment progression (FIGS. 2 and 8).

In various embodiments, the processor 701 can be a standard central processing unit or a secure processor. Secure processors can be special-purpose processors (e.g., reduced instruction set processor) that can withstand sophisticated attacks that attempt to extract data or programming logic. The secure processors may not have debugging pins that enable an external debugger to monitor the secure processor's execution or registers. In other embodiments, the system may employ a secure field programmable gate array, a smartcard, or other secure devices.

The memory 702 can be standard memory, secure memory, or a combination of both memory types. By employing a secure processor and/or secure memory, the system can ensure that data and instructions are both highly secure and sensitive operations such as decryption are shielded from observation. The memory 702 can contain executable instruction for cooling the surface of the subject's skin to a temperature and controlling treatment devices in response to, for example, detection of a or total freeze event. The memory 702 can include thawing instructions that, when executed, causes the controller to control the applicator to heat tissue. In some embodiments, the memory 702 stores instructions that can be executed to control the applicators to perform the methods disclosed herein without causing undesired effects, such as significantly lightening or darkening skin one of more days after the freeze event ends. The instructions and treatment programs can be modified based on patient information, treatments to be performed, or other treatment parameters. The instructions can be executed to perform the methods disclosed herein.

Suitable computing environments and other computing devices and user interfaces are described in commonly assigned U.S. Pat. No. 8,275,442, entitled “TREATMENT PLANNING SYSTEMS AND METHODS FOR BODY CONTOURING APPLICATIONS,” which is incorporated herein in its entirety by reference.

### G. Conclusion

It will be appreciated that some well-known structures or functions may not be shown or described in detail, so as to avoid unnecessarily obscuring the relevant description of the various embodiments. Although some embodiments may be within the scope of the technology, they may not be described in detail with respect to the Figures. Furthermore, features, structures, or characteristics of various embodiments may be combined in any suitable manner. The technology disclosed herein can be used for improving the appearance of skin and to perform the procedures disclosure in U.S. Provisional Application Ser. No. 61/943,250, filed Feb. 21, 2014, U.S. Pat. No. 7,367,341 entitled “METHODS AND DEVICES FOR SELECTIVE DISRUPTION OF FATTY TISSUE BY CONTROLLED COOLING” to Anderson et al., and U.S. Patent Publication No. US 2005/0251120 entitled “METHODS AND DEVICES FOR DETECTION AND CONTROL OF SELECTIVE DISRUPTION OF FATTY TISSUE BY CONTROLLED COOLING” to Anderson et al., the disclosures of which are incorporated herein by reference in their entireties. The technology disclosed herein can target tissue for tightening the skin, improving skin tone or texture, eliminating or reducing wrinkles, increasing skin smoothness as disclosed in U.S. Provisional Application Ser. No. 61/943,250.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Use of the word “or” in reference to a list of two or more items covers all of the following interpretations of the word: any of the items in the list, all of the items in the list, and any combination of the items in the list. In those instances where a convention analogous to “at least one of A, B, and C, etc.” is used, in general such a construction is intended in the sense of the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense of the convention (e.g., “a system having at least one of A, B, or C” would include but not be limited to systems that have A alone, B alone, C alone, A and B together, A and C together, B and C together, and/or A, B, and C together, etc.).

Any patents, applications and other references, including any that may be listed in accompanying filing papers, are incorporated herein by reference. Aspects of the described technology can be modified, if necessary, to employ the systems, functions, and concepts of the various references described above to provide yet further embodiments. While the above description details certain embodiments and describes the best mode contemplated, no matter how detailed, various changes can be made. Implementation details may vary considerably, while still being encompassed by the technology disclosed herein. The various aspects and embodiments disclosed herein are for purposes of illustration and are not intended to be limiting, with the true scope and spirit being indicated by the following claims.

What is claimed is:

1. A method for treating a patient, comprising:
cooling a surface of the patient's skin to cool tissue under the surface to a temperature between −40 degrees C. and 0 degrees C. to damage targeted tissue; and
applying electromagnetic energy or an electromagnetic field to the patient to inhibit freezing of the patient's tissue while the targeted tissue cooled via cooling of the surface is supercooled.

2. The method of claim 1, further comprising applying the electromagnetic energy or field while the targeted tissue is damaged due to the cooling of the surface.

3. The method of claim 1, further comprising cooling the surface of the patient's skin for a length of time sufficient to selectively affect the targeted tissue, thereby reducing skin surface irregularities.

4. The method of claim 1, further comprising cooling the surface of the patient's skin for a length of time sufficient to cause damage to lipid rich cells in the patient's subcutaneous layer.

5. The method of claim 1, wherein the electromagnetic field is applied and is strong enough to inhibit freezing of liquid and/or lipids in the patient's tissue.

6. The method of claim 1, wherein a subcutaneous region of the patient is kept in a supercooled state for a period of time, wherein the electromagnetic energy or field is applied during at least a portion of the period of time.

7. The method of claim 1, wherein a sufficient amount of electromagnetic energy is delivered to the skin to protect one or more layers of the subject's tissue.

8. The method of claim 1, further comprising cooling the surface of the patient's skin such that subcutaneous lipid-rich cells are damaged.

9. The method of claim 1, wherein the electromagnetic field is an AC or DC electric field.

10. A method for treating a patient, comprising:
applying an applicator to the patient's body;
cooling a surface of the patient's skin with the applicator to cool targeted tissue beneath the surface to a temperature between −40 degrees C. and 0 degrees C.; and
applying electromagnetic energy or an electromagnetic field with the applicator to inhibit freezing of at least a portion of the tissue below the cooled surface such that the patient's tissue is in a supercooled state.

11. The method of claim 10, wherein the patient's subcutaneous tissue is in the supercooled state while the electromagnetic energy or field is applied.

12. The method of claim 10, further comprising applying the electromagnetic energy or field prior to the targeted tissue becoming damaged due to the cooling of the surface.

13. The method of claim 10, further comprising delivering a sufficient amount of the electromagnetic energy to inhibit freeze injury to shallow tissue cooled by the applicator while allowing thermal injury to deeper tissue.

14. The method of claim 10, wherein the electromagnetic energy or field is applied while the targeted tissue is at a temperature lower than −5 degrees C.

15. The method of claim 10, wherein the electromagnetic energy or field is continuously applied for a predetermined period of time.

16. The method of claim 10, wherein the electromagnetic energy or field is periodically applied while cooling the surface.

17. The method of claim 10, further comprising delivering a cryoprotectant to the surface of the patient's skin to protect the subject's epidermal tissue.

18. The method of claim 10, further comprising controlling the applicator to cool the patient's skin such that ice crystals are present in the skin for a sufficient length of time to cause a reduction in the skin irregularities without causing necrosis.

19. The method of claim 10, wherein the method improves the appearance of cellulite, improves the appearance of skin, improves skin tone and texture, thickens skin, eliminates deep skin wrinkles, eliminates fine lines in the skin, tightens skin, and/or treats sweat glands.
